(19) United States
(12) Patent Application Publication
(10) Pub. No.: US 2012/0244169 A1
(43) Pub. Date: Sep. 27, 2012

(54) Treatment for Radiation-Induced Disorders

(75) Inventor: Kenneth E. Lipson, San Mateo, CA (US)

(73) Assignee: FibroGen, Inc., San Francisco, CA (US)

(21) Appl. No.: 13/508,524

(22) PCT Filed: Nov. 8, 2010
(86) PCT No.: PCT/US10/02922

§ 371 Date: Jun. 12, 2012

Related U.S. Application Data
(60) Provisional application No. 61/280,634, filed on Nov. 6, 2009.

(51) Int. Cl. A61K 39/395; A61P 9/00; A61P 17/00; A61P 1/00; A61P 13/12; C07K 16/22; A61P 11/00

(52) U.S. Cl. 424/158.1; 530/389.2

(57) ABSTRACT

The present invention relates to methods and medicaments useful for treatment of radiation-induced disorders by administering anti-CTGF agents, particularly anti-CTGF antibodies. Methods and medicaments for pre-treating individuals having or at risk for having exposure to ionizing radiation to prevent or reduce radiation-induced disorders are also provided.

This application claims the benefit under 35 U.S.C. 119(e) of U.S. provisional application Ser. No. 61/280,634, filed on 6 Nov. 2009, which application is incorporated by reference herein in its entirety.

### FIELD OF THE INVENTION

The present invention relates to methods and medicaments useful for treatment of radiation-induced disorders. Methods and medicaments for treating or pre-treating individuals having or at risk for having exposure to ionizing radiation to prevent, reduce or stabilize radiation-induced disorders are also provided.

### BACKGROUND

The most significant source of ionizing radiation in the general public is from medical procedures such as diagnostic X-rays, nuclear medicine, and radiation therapy. In addition, occupational exposure to radiation is a concern in certain industries including airline travel, mining, industrial radiography, nuclear energy production, and research laboratories, where individuals are at a higher risk of exposure.

Radiation therapy, which uses ionizing radiation to kill cancer cells and shrink tumors, is used in treating approximately half of all people with cancer. Radiation therapy is a component of curative therapy for a number of diseases including breast cancer, Hodgkin's disease, head and neck cancer, prostate cancer, and gynecological cancers. In high risk settings, radiation therapy can prevent the development of leptomeningeal disease and brain metastases in acute leukemia and lung cancer. With respect to lung cancer, for example, both small-cell and non-small cell lung cancers are frequently treated with radiation therapy, which may be used alone or in combination with chemotherapy, surgery or both. More than half of patients diagnosed with non-small cell lung cancer will receive radiation therapy at some time during their treatment. Although radiation therapy is a proven method for controlling malignancies and prolonging life of the cancer patient, radiation also causes injury to normal tissues. Irradiation of cancers inevitably leads to irradiation of some normal tissue which can then give rise to tissue injury and loss of function. Side effects of radiation therapy, both acute and chronic, are well known in many organs. Severe reactions in a small number of patients impose limits on the radiation dose that can be delivered, not just to those patients individually but to any patient. Approximately 5-20% of patients treated for lung cancer with radiation therapy develop symptomatic lung injury due to irradiation, while 50-90% experience declines in pulmonary function. Thus, radiation-induced pulmonary disorders are a major limiting factor in the successful treatment of thoracic tumors. Radiation enteritis has been estimated to occur in up to 20% of patients receiving abdominal or pelvic radiation therapy (See, e.g., Theis et al. (2010) Clin Oncol. (R Coll Radiol) 22:70-83). Radiation therapy is the main cause of pericarditis and pericardial effusion, in all overall 30% incidence of clinically detectable heart injury, after thoracic or mediastinal irradiation. (See, e.g., Galderisi et al. (2007) Cardiovascular Ultrasound 5:4) Radiation esophagitis is a common side effect of radiation therapy for head and neck cancer or lung cancer. The ability to effectively treat cancer using radiation therapy requires that the radiation dose be sufficient to kill the tumor while minimizing the deleterious effect on normal tissues surrounding the tumor. (See, e.g., Hendry et al. (2006) Pan Am J Public Health 20:151-160; Movsas et al. (1997) Chest 111:1061-1076.)

Various strategies have been used in an attempt to increase radiation dose while having minimal impact on normal tissue. Current methods are directed at reducing the irradiated volume and applying multiple small dose fractions of ionizing radiation that allow normal tissue to recover in between doses. Agents that increase the susceptibility of the tumor to the damaging effects of radiation (radiosensitizers) are also being tested, as are agents that increase resistance of normal tissue to the effects of radiation (radioprotectors). However, as the damaging side effects of radiation on the long-term function of irradiated organs remain problematic, new methods and medicaments for the treatment of radiation-induced disorders are desirable. In particular, methods and medicaments for treating individuals having or at risk for having a radiation-induced disorder as a result of exposure to radiation therapy are also desirable.

### SUMMARY OF THE INVENTION

The present invention provides methods and agents for treating a radiation-induced disorder in a subject having, or at risk of having, a radiation-induced disorder, the method comprising administering to the subject an anti-connective tissue growth factor (anti-CTGF) agent. The radiation-induced disorder typically results from ionizing radiation exposure of the subject. In one aspect the exposure of the subject to ionizing radiation is a consequence of radiation therapy. In other aspects the exposure of the subject to ionizing radiation results from a known or suspected occupational or environmental exposure to ionizing radiation.

In particular aspects the radiation-induced disorder can be a disorder induced by irradiation of any, or multiple, body parts, organs or tissues of the subject, including but not limited to lung, heart, bladder, gastrointestinal tract, large intestine, small intestine, stomach, esophagus, skin, ovaries, testes, urogenital system, kidney, head, neck, pancreas, liver, brain, spinal cord, prostate, vasculature, and muscle. In various aspects the radiation-induced disorder can be one or more of radiation pneumonitis, radiation enteritis, radiation enteropathy, radiation enterocolitis, radiation dermatitis, radiation-induced erythema, radiation colitis, radiation proctitis, radiation cystitis, radiation nephritis, radiation esophagitis, radiation pericarditis, radiation-induced cardiac effusion, and radiation-induced cardiac fibrosis. The methods and agents of the present invention are effective for treating, preventing, reducing, stabilizing, or reversing pathological features associated with radiation-induced disorders. In one aspect the invention provides a method of, and an agent for, treating, preventing, reducing, stabilizing, or reversing a pathological feature associated with a radiation-induced disorder. Such pathological features are well-known and may vary depending upon the particular radiation-induced disorder.

The method and agents of the present invention are particularly effective for the treatment of a radiation-induced disorder of the lung and/or heart. The method and agents of the present invention are particularly effective for the treatment of a radiation pneumonitis, radiation pericarditis, radiation-induced cardiac effusion, and/or radiation-induced cardiac fibrosis. In one aspect the invention provides a method of, and an agent for, treating a radiation-induced disorder of the lung. In another aspect the invention provides a method of, and an agent for, treating, a radiation-induced disorder of the heart. In a separate aspect, the invention provides a method of; and agent for, treating a radiation-induced disorder selected from the group of radiation pneumonitis, radiation pericarditis, radiation-induced cardiac effusion, and radiation-induced cardiac fibrosis.

In one aspect the present invention provides a method of, and an agent for, improving lung function in a subject having an impaired lung function resulting from a radiation-induced disorder, the method comprising administering to the subject an anti-CTGF agent, thereby improving lung function in the subject.

In one embodiment, the methods and agents of the present invention are particularly effective for treating, preventing, reducing, stabilizing, or reversing pathological features associated with a radiation-induced lung disorder that contribute to impaired lung function. Exemplary pathological features associated with a radiation-induced lung disorder include increased lung density, decreased fraction potential airspace, decreased lung volume, increased lung tissue remodeling, and decreased PaO₂. In particular aspects of this embodiment, the methods and agents of the present invention are effective for treating, preventing, reducing, stabilizing, or reversing the increased lung density associated with radiation-induced lung disorder, the decreased fraction potential airspace associated with radiation-induced lung disorder, the decreased lung volume associated with radiation-induced lung disorder, the increased lung tissue remodeling associated with radiation-induced lung disorder, and/or the decreased PaO₂ associated with radiation-induced lung disorder.

In another aspect the present invention provides a method of, and an agent for, reducing, reversing, or stabilizing lung density in a subject having increased lung density associated with a radiation-induced lung disorder, the method comprising administering to the subject an anti-CTGF agent, thereby reducing, reversing, or stabilizing lung density in the subject.

In another aspect the present invention provides a method of, and an agent for, reducing, reversing, or stabilizing lung remodeling in a subject having increased lung remodeling associated with a radiation-induced lung disorder, the method comprising administering to the subject an anti-CTGF agent, thereby reducing or stabilizing lung remodeling in the subject.

In yet another embodiment, the present invention provides a method of, and an agent for, increasing the likelihood of survival in a subject having a radiation-induced disorder, the method comprising administering to the subject an anti-CTGF agent, thereby increasing the likelihood of survival in the subject. In a particular embodiment, the subject has a radiation-induced lung disorder.

The methods of the present invention are accomplished by administration of an anti-CTGF agent to a subject having, or at risk of having, a radiation-induced disorder. The administration of the anti-CTGF agent is carried out by methods that are well-known and are described in detail herein. The anti-CTGF agent is one that specifically and directly inhibits the activity of the connective tissue growth factor (CTGF) protein or expression of the CTGF gene. In particular embodiments the anti-CTGF agent is an antibody, particularly a monoclonal antibody, that binds specifically to CTGF protein, or a polynucleotide inhibitor of CTGF expression (for example, a CTGF antisense oligonucleotide, siRNA, shRNA, or miRNA). In a preferred embodiment, the anti-CTGF agent is an antibody that binds specifically to CTGF. In various embodiments, the anti-CTGF agent is an antibody described and claimed in Lin et al., United States Patent Application Publication No. 2009/0017043 or in U.S. Pat. No. 7,405,274, which application and patent are incorporated by reference herein. In particular embodiments, the anti-CTGF agent is an antibody that has the amino acid sequence of the antibody produced by the cell line identified by ATCC Accession No. PTA-6006. In other embodiments, the anti-CTGF agent is an antibody that binds to CTGF competitively with an antibody produced by ATCC Accession No. PTA-6006. A particular antibody for use as anti-CTGF agent in the present methods is CLN1 or mAb1 as described in U.S. Pat. No. 7,405,274, or an antibody substantially equivalent thereto or derived therefrom.

The invention provides anti-CTGF agents for use in the methods described herein. The invention provides anti-CTGF agents for use in preparation of medicaments for use in the methods described herein. The invention provides anti-CTGF agents for treatment of radiation-induced disorders in a subject. The invention provides anti-CTGF agents for treatment of radiation-induced disorders in a subject resulting from ionizing radiation exposure of the subject. The invention provides anti-CTGF agents for treatment of radiation-induced disorders in one or more of the lung, heart, bladder, gastrointestinal tract, large intestine, small intestine, stomach, esophagus, skin, ovaries, testes, urogenital system, kidney, head, neck, pancreas, liver, brain, spinal cord, prostate, vasculature, and/or muscle of a subject resulting from ionizing radiation exposure of the subject. The invention provides anti-CTGF agents for treatment of one or more of radiation pneumonitis, radiation enteritis, radiation enteropathy, radiation enterocolitis, radiation dermatitis, radiation-induced erythema, radiation colitis, radiation proctitis, radiation cystitis, radiation nephritis, radiation esophagitis, radiation pericarditis, radiation-induced cardiac effusion, and/or radiation-induced cardiac fibrosis in a subject. The invention provides anti-CTGF agents for improvement of lung function in a subject having impaired lung function resulting from a radiation-induced disorder. The invention provides anti-CTGF agents for reducing, reversing, or stabilizing lung density in a subject having a radiation-induced lung disorder.

These and other embodiments of the present invention will readily occur to those of skill in the art in light of the disclosure herein, and all such embodiments are specifically contemplated.

### BRIEF DESCRIPTION OF THE DRAWINGS

FIG. 1 sets forth various aspects of an animal model of radiation-induced pulmonary disorder. FIG. 1A provides a schematic of the different dosing schedules exemplified in the examples herein for the present methods and medicaments following exposure to ionizing radiation. FIG. 1B sets forth the typical course of leukocyte infiltration following exposure to ionizing radiation. FIG. 1C sets forth the typical course of edema and fibrosis following exposure to ionizing radiation. FIG. 1D sets forth the typical course of changes in lung density following exposure to ionizing radiation.

FIG. 2 shows the fraction potential airspace in the lungs of mice treated with anti-CTGF antibody beginning at various times before or after exposure to ionizing radiation. FIG. 2A shows the fraction potential airspace present over time in the lungs of mice when administration of anti-CTGF antibody was initiated 2 days before (IR αCTGF mAb d−2) or 2 days after (IR αCTGF mAb d+2) the exposure to ionizing radiation and treatment continued for 8 weeks. FIG. 2B shows the fraction potential airspace present over time in the lungs of mice when administration of anti-CTGF antibody was initiated 20 days after (IR αCTGF mAb d+20) the exposure to ionizing radiation and treatment continued for 8 weeks. FIG. 2C shows the fraction potential airspace present over time in the lungs of mice when administration of the anti-CTGF antibody was initiated 112 days (IR αCTGF mAb d+112) after the exposure to ionizing radiation and treatment continued for 8 weeks. For each panel, controls of irradiated but untreated mice (IR) and unirradiated but treated with anti-CTGF antibody (CTGF mAb) mice are also shown.

FIG. 3 shows the increase in lung density of mice following irradiation and the effect of anti-CTGF antibody on the lung density increase. Lung density is measured in a scale of Hounsfield units (HU): +1000 HU is the density of very dense tissue like bone, 0 HU is the density of water and −1000 HU is the density of air. FIG. 3A shows the lung densities over time for mice treated with anti-CTGF antibody beginning 2 days before (FR αCTGF mAb d−2) or 2 days after (IR αCTGF mAb d+2) the exposure to ionizing radiation and treatment continued for 8 weeks. FIG. 3B shows the lung densities of mice treated with anti-CTGF antibody beginning 20 days after (ER αCTGF mAb d+20) the exposure to ionizing radiation and treatment continued for 8 weeks. FIG. 3C shows the lung densities of mice treated with anti-CTGF antibody beginning 112 days after (IR αCTGF mAb d+112) the exposure to ionizing radiation and treatment continued for 8 weeks. In each panel, the lung densities of control mice that were either irradiated and untreated (IR) or unirradiated and treated with anti-CTGF antibody (αCTGF mAb) are shown for comparison.

FIG. 4 shows the partial pressure O₂ and blood oxygen saturation percent in blood sample taken from tail capillaries from mice in each treatment group at 30 weeks after irradiation. FIG. 4A shows the average blood partial pressure O₂ (PaO₂) for each treatment group. The striped box in the figure defines the normal range for PaO₂ in this model organism. FIG. 4B plots the average blood oxygen saturation percent (filled circles) and inverse lung density (filled squares) for each treatment group, showing that improved lung function (greater oxygen saturation) correlates inversely with lung density. The shaded region in the figure defines the normal oxygen saturation percent range in this model organism. The treatment groups are indicated as in FIG. 2. The “IgG” and “IR+IgG” groups are unirradiated and treated with IgG, and irradiated and treated with IgG, respectively.

FIG. 5 sets forth the survival rate for mice treated with the methods and medicaments of the invention. FIG. 5A shows the percent of mice surviving at various times from the treatment groups in which anti-CTGF antibody administration was initiated 2 days before or 2 days after the exposure to ionizing radiation and treatment continued for 8 weeks. The treatment period is indicated at the top of each graph. FIG. 5B shows the percent of mice surviving at various times from the treatment group in which anti-CTGF antibody administration was initiated 20 days after the exposure to ionizing radiation and treatment continued for 8 weeks. FIG. 5C shows the percent of mice surviving at various times from the treatment groups in which anti-CTGF antibody administration was initiated 112 days after the exposure to ionizing radiation and treatment continued for 8 weeks. All of the treatment groups are indicated as in FIG. 2. For comparison, each panel shows the percent of mice surviving in the irradiated and untreated group (IR) and in the unirradiated and treated with anti-CTGF antibody group (αCTGF mAb). All of the irradiated groups treated with anti-CTGF antibody showed better survival rates than the irradiated and untreated group.

FIG. 6 shows Sirius Red stained heart sections, left and right ventricles, from mice that were irradiated and untreated (IR), unirradiated and treated with IgG (IgG) or irradiated and treated with anti-CTGF antibody beginning at 20 days after irradiation (IR+anti-CTGF Ab 20 d Post). The darker staining areas, particularly seen in the IR sections, are indicative of collagen deposition. Very little, if any, collagen staining is seen in the heart sections from the unirradiated mice treated with IgG or in the irradiated mice treated with anti-CTGF antibody beginning at 20 days after irradiation.

FIG. 7 shows the quantitation of collagen staining of the heart cross sections seen in FIG. 6. To quantify collagen deposition, photographs of sections of the right and left ventricles were examined with Image-Pro Plus software (version 6.1; Media Cybernetics Inc., Bethesda, Md.) and the area represented by intense staining was expressed as a percent of total area.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

It must be noted that as used herein and in the appended claims, the singular forms “a,” “an,” and “the” include plural references unless context clearly dictates otherwise. Thus, for example, a reference to “a fragment” includes a plurality of such fragments; a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the present invention. Nothing herein is to be construed as an admission that the present invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

In one embodiment, the present invention provides a method of, and agents and medicaments for, treating a radiation-induced disorder in a subject having, or at risk of having, a radiation-induced disorder, the method comprising administering to the subject an anti-connective tissue growth factor (anti-CTGF) agent, thereby treating the disorder. Typically, the radiation-induced disorder results from ionizing radiation exposure in the subject.

A radiation-induced disorder is any disorder, disease, or pathological condition that occurs as a result of, or is induced by, exposure of a subject to ionizing radiation of sufficient intensity and duration to bring about an undesirable effect, for example, undesirable tissue damage. The amount of ionizing radiation exposure that results in radiation-induced disorders intended to be treated by the methods and agents herein is generally between the minimal tolerance dose and the maximal tolerance dose. The minimal tolerance dose (T/D_{5/5}) is the dose that when administered to a given patient population under a standard set of treatment conditions, results in a rate of severe complications of 5% or less within 5 years of treatment. The maximal tolerance dose (T/D_{50/5}) is the dose that when administered to a given patient population under a standard set of treatment conditions, results in a rate of severe complications of 50% or less within 5 years of treatment. T/D_{5/5} and T/D_{50/5} have been established for many conditions and are well-known (see, e.g., Rubin et al. (Eds) Radiation Biology and Radiation Pathology Syllabus, set RT 1 Radiation Oncology, Chicago, American College of radiology, 1975). The minimal tolerance dose and maximal tolerance dose have been established with respect to therapeutic radiation treatments but are applicable as well for determining the range of radiation exposure suitable for causing the radiation-induced disorders resulting from exposure to radiation from other sources (e.g., occupational or environmental exposures). Radiation is quantitated on the basis of the amount of radiation absorbed by the body, not based on the amount of radiation produced by the source. A rad (radiation absorbed dose) is 100 ergs of energy per gram of tissue; a gray (Gy) is 100 rad. Radiation dose can be measured by placing detectors on the body surface or by calculating the dose based on radiating phantoms that resemble human form and substance. Radiation dose has three components: total absorbed dose, number of fractions, and time. Most teletherapy radiation therapy programs are fractionated, being delivered being delivered in fractions periodically over time, typically once a day, 5 days a week, in 150-200 cGy fractions, generally applied to limited target areas of the body. The total dose delivered in radiation therapy will vary depending on the nature and severity of the condition being treated. For curative cases, the absorbed dose typically will range from 20-80 Gy. For preventative cases, doses are typically around 45-60 Gy and are applied in fractions of about 1.8-2 Gy per day. When used for radiation therapy, ionizing radiation is usually provided over a period of time or until a particular amount of radiation exposure has been reached by the target area of the subject.

Sources of ionizing radiation include electrons, X-rays, gamma rays, and atomic ions. Exposure of a subject to ionizing radiation may be due to a medical procedure including, but not limited to, radiation therapy to treat certain malignant conditions, e.g., lung or breast cancer; medical procedures such as diagnostic X-rays; or procedures involving administration of nuclear medicines. Exposure to ionizing radiation can also come from known or suspected occupational or environmental sources, e.g., various consumer products including, but not limited to, tobacco, combustible fuels, smoke detectors, and building materials, or as a consequence of a nuclear accident. Typically, in the radiation-induced disorders suitable for treatment with methods of the present invention, the source of the ionizing radiation is radiation therapy.

Radiation therapy is the medical use of high-energy ionizing radiation to shrink tumors and control malignant cell growth. Radiation therapy also has some application to non-malignant conditions, for example treatment of trigeminal neuralgia, severe thyroid eye disease, pterygium, pigmented villonodular synovitis, or prevention of keloid scar growth, but its use is limited because of concerns related to risk of radiation induced cancers. X-rays, gamma rays, and charged particles are types of radiation used for radiation therapy. The radiation may be delivered by a machine outside the body (external-beam radiation therapy, also called teletherapy), or it may come from encapsulated radioactive material implanted directly into or adjacent to tumor tissues in the body near cancer cells (internal radiation therapy, also called brachytherapy). Systemic radiation therapy uses radioactive substances, such as radioactive iodine, that travel in the blood and are targeted in some fashion to the cancer cells. Teletherapy is the most common form of radiation therapy. About half of all cancer patients receive some type of radiation therapy sometime during the course of their treatment.

Radiation-induced disorders in different tissues and organs generally follow a similar course after exposure to ionizing radiation, particularly as a consequence of radiation therapy. An acute response phase occurs from several days to several months following exposure to ionizing radiation which involves inflammatory components, is generally self-limiting, and appears to resolve within a relatively short time. The acute phase is often followed by a chronic phase which generally occurs beginning at about several months up to several years after exposure. The chronic phase is often characterized by extensive tissue remodeling and fibrosis. Results presented herein suggest that effective treatment of the acute response may mitigate or attenuate the chronic phase. Cancers or tumors that occasionally develop, often many years later, at or near the site of radiation exposure are not intended to be included among the disorders suitable for treatment in the method of the present invention.

Radiation-induced disorders, particularly those resulting from radiation therapy, are well known and have been observed in a variety of tissues and organs. The radiation-induced disorder is not the intended result of the radiation therapy but rather is an unintended, and undesirable, side effect of the exposure of various organs, tissues and body parts to the ionizing radiation used in radiation therapy. The radiation-induced disorder can be a disorder induced by irradiation of any, or multiple, body parts, organs or tissues of the subject, including but not limited to lung, heart, bladder, gastrointestinal tract, large intestine, small intestine, stomach, esophagus, skin, ovaries, testes, urogenital system, kidney, head, neck, pancreas, liver, brain, spinal cord, prostate, vasculature, and muscle. In various aspects the radiation-induced disorder can be, but is not limited to one or more of radiation pneumonitis, radiation enteritis, radiation enteropathy, radiation enterocolitis, radiation dermatitis, radiation-induced erythema, radiation colitis, radiation proctitis, radiation cystitis, radiation nephritis, radiation esophagitis, radiation pericarditis, radiation-induced cardiac effusion, and radiation-induced cardiac fibrosis. All of these disorders are well-known and readily identifiable by competent medical practitioners.

For example, radiation enteritis (including radiation enterocolitis, radiation colitis, radiation proctitis) has been estimated to occur in 2-5% of patients receiving pelvic or abdominal radiation therapy. Pathological changes of the intestinal epithelial layer occur early in radiation treatments. Within 12-24 hours of the first radiation dose, pathological evidence of dead cells can be seen in the mucosal lining. Patients often experience symptoms similar to other forms of acute gastritis, including epigastric pain, loss of appetite, nausea, and vomiting. Symptoms of the acute response generally resolve within a few weeks but manifestation of chronic response can occur between 6 months and up to 5 years following radiation exposure. Progressive fibrosis, perforation, fistula formation, and stenosis of the irradiated portion of the intestine can occur during the chronic phase of radiation enteropathy.

Radiation injury to the bladder (radiation cystitis) generally becomes symptomatic 3 to 6 weeks after radiation exposure, with patients complaining of increased frequency and dysuria. Diffuse mucosal changes, as well as desquamation or ulceration may be observed. Chronic stage effects of radiation cystitis can include interstitial fibrosis, telangiectasia, and ulceration, as well as dilation and rupture of blood vessels, resulting in hematuria.

Radiation nephritis or radiation nephropathy may occur after irradiation of one or both kidneys, and can result in kidney failure. Radiation nephritis can occur as a result of bone marrow transplantation procedures or as a result of exposure of the kidneys when radionuclides used in radiotherapy (systemic radiation therapy) are filtered through the kidneys and reabsorbed by the renal tube epithelium. All components of the kidney can be affected by radiation nephritis, including the glomeruli, blood vessels, tubular epithelium, and interstitium. Symptoms in the acute phase, which typically occur within 6-12 months after radiation, can include proteinuria and hypertension, but acute radiation nephritis may be asymptomatic. Chronic phase response may include progressive scarring of the irradiated kidney and severe hypertension.

Subjects having a radiation-induced lung disorder (e.g., radiation pneumonitis) typically present with a nonproductive cough, shortness of breath, and low-grade fever. Upon evaluation, subjects are found to have reduced total lung volume, residual volume, and vital capacity, but unrestricted air flow into and out of the lungs. Diagnosis of a radiation-induced lung disorder is based on symptoms including dyspnea, nonproductive cough, and low-grade fever; and generally involves blood tests, e.g., measurement of partial oxygen saturation of the blood; pulmonary function tests, e.g., measurement of total lung volume, residual volume, and vital capacity; and computed tomography (CT) scans of the thorax, e.g., to measure lung density and monitor lung remodeling.

Acute pericarditis may result from cardiac irradiation. The symptoms can include chest pain and fever, with or without pericardial effusion, and typically manifest within a few months after irradiation. Asymptomatic pericardial effusion may be the most common manifestation of radiation-induced heart disorder. It is usually detected by chest x-ray and confirmed by echocardiogram. Patients receiving larger radiation doses may experience symptomatic constrictive pericarditis. Chronic cardiac effects may have their onset from 6 months to several years following irradiation. Clinical symptoms may include chronic constrictive disease due to pericardial, myocardial, and endocardial fibrosis, exhibiting signs such as dyspnea, chest pain, venous distention, pleural effusion and paradoxical pulse.

Radiation related esophageal complications (radiation esophagitis) are common side effects of therapeutic radiation to the neck, chest, or mediastinum. Radiation related complications typically can occur in 5% of patients exposed to 6000 rads in the esophageal window and 25%-50% of patients exposed to 7500 rads though the incidence of radiation induced toxicity can vary based on dosimetry, differences in radiation technique, and potentially radiosensitizing hemotherapy. Patients can develop acute radiation esophagitis with symptoms of substernal burning, dysphagia and odynophagia within 3 weeks following exposure. Chronic radiation esophagitis is a consequence of the submucosal fibrosis and chronic arteriolitis. It is characterized histologically by marked thickening of the esophageal wall, expansion of the submucosa, and increased endothelial proliferation. This process may lead to stricture formation, and less commonly, complications such as perforation and fistulization.

In the methods of the invention for treating a radiation-induced disorder, “treating” the disorder intends administering a therapeutic (e.g., anti-CTGF agent) to the subject in order to achieve a beneficial effect on the radiation-induced disorder, including on the symptoms, pathological features, consequences, or adverse effects of the radiation-induced disorder. Treating may additionally include pre-treating or preventing the disorder. Treating the disorder may be effected by reducing, stabilizing or reversing the disorder, or the symptoms, pathological features, consequences, or adverse effects of the disorder. Pre-treating the disorder includes initiation of the administration of a therapeutic (e.g., an anti-CTGF agent) at a time prior to the appearance or existence of the radiation-induced disorder, or prior to the exposure of a subject to ionizing radiation. Pre-treating the disorder is particularly applicable to subjects at risk of having a radiation-induced disorder. Preventing the radiation-induced disorder intends initiation of the administration of a therapeutic (e.g., an anti-CTGF agent) at a time prior to the appearance or existence of the radiation-induced disorder such that the radiation-induced disorder, or its symptoms, pathological features, consequences, or adverse effects do not occur. By stabilizing the radiation-induced disorder is intended that the radiation-induced disorder, or its symptoms, pathological features, consequences, or adverse effects do not substantially worsen after administration of the therapeutic to the subject. By reducing the radiation-induced disorder is intended that the radiation-induced disorder, or its symptoms, pathological features, consequences, or adverse effects are less deleterious than expected by comparison with untreated subjects (i.e., subjects having a radiation-induced disorder but untreated with the anti-CTGF agents of the present invention). By reversing the radiation-induced disorder is intended that the radiation-induced disorder, or its symptoms, pathological features, consequences, or adverse effects are less severe after administration of the therapeutic than prior to administration of the therapeutic (i.e., less severe after treatment than prior to treatment).

When the methods of the invention are used prior to exposure to ionizing radiation, the methods may additionally decrease the sensitivity of normal tissue to ionizing radiation. As provided herein, pretreatment using the methods of the invention prior to thoracic exposure to ionizing radiation improved lung density and lung function, and improved survival rates in subjects subsequent to exposure. The invention therefore contemplates that when the methods of the invention are applied prior to exposure, the subject could receive a higher level of radiation exposure without significant loss of lung function. This is particularly useful in radiation therapy, where the methods of the invention can be used to pre-treat a subject prior to exposure (e.g., thoracic exposure) to ionizing radiation, thereby allowing higher doses of radiation and/or more frequent dosing of radiation without adversely affecting organ function (e.g., lung function) and survivability of the subject. The ability to apply higher doses of radiation or more frequent dosing would improve the likelihood of eradicating the tumor.

The methods and agents of the present invention are effective for treating, preventing, reducing, stabilizing, or reversing a pathological feature associated with radiation-induced disorders. In one aspect the invention provides a method of, and an agent for, treating, preventing, reducing, stabilizing, or reversing a pathological feature associated with a radiation-induced disorder. Such pathological features are well-known and may vary depending upon the particular radiation-induced disorder. For example, exemplary pathological features associated with a radiation-induced lung disorder include impaired lung function, increased lung density, increased lung remodeling, decreased fraction potential airspace, decreased PaO₂, decreased lung volume, decreased lung vital capacity, increased septal thickness, increased leukocyte infiltration, and elevated percentage of polymorphonuclear leukocytes (PMNs) in BAL fluid. Pathological feature associated with other radiation-induced disorders are well-known and many of these are discussed infra.

In one embodiment, the invention provides a method of treating a radiation-induced disorder wherein the radiation-induced disorder results in impaired lung function and the method improves the impaired lung function. By impaired lung function is intended that the lung function in the subject having the radiation-induced disorder is lower (by any measure of lung function) than prior to having the radiation-induced disorder. Alternatively, the impaired lung function in the subject can be evaluated and compared to a standard measure of lung function for a matched control. In this case, the impaired lung function will be lower than the standard measure. Impaired lung function can be determined by any method that is usual and customary for evaluating lung function including measurement of blood gas parameters, e.g., partial arterial pressure of oxygen (PaO₂) or percent oxygen saturation of blood, diffusing capacity in the lung of CO, measurement of lung volume parameters, e.g., lung vital capacity and/or total lung volume, or measurement of the cellular make-up of bronchoalveolar lavage (BAL) fluid, measurement of the lung density, measurement of the fraction potential airspace in the lung, measurement of lung tissue remodeling, measurement of deposition of ECM, determination of pneumonitis. The impaired lung function is improved when the lung function moves closer to the level of lung function in the standard measure or in the subject prior to the radiation-induced disorder. In various embodiments, the methods of the present invention are used to treat a subject having a PaO₂ of below 80 mmHg, particularly below 75 mmHg, and more particularly below 70 mmHg. In some embodiments, lung function is determined by measuring lung volume parameters, e.g., lung vital capacity and/or total lung volume. For example, improved lung function can be determined in subjects having low lung vital capacity by measuring the ability of the present methods to increase lung vital capacity. In certain embodiments wherein the subject has below normal lung vital capacity and/or total lung volume, the methods increase lung vital capacity and/or total lung volume. In some embodiments, lung function is determined by measuring the cellular make-up of bronchoalveolar lavage (BAL) fluid. In certain embodiments, the methods of the invention normalize the cellular make-up of BAL fluid. In various embodiments, the methods of the present invention are used to treat a subject having an elevated percentage of polymorphonuclear leukocytes (PMNs) in BAL fluid and the methods of the invention reduce the percentage of PMNs in BAL fluid. In particular embodiments, the subject has greater than 5% PMNs in BAL fluid, particularly greater than 10%, and more particularly greater than 15%. In particular embodiments, lung function is normalized over the treatment time course.

Thus, in particular embodiment, the present invention provides a method of treating a radiation induced disorder, wherein the disorder results in impaired lung function, and the method improves the impaired lung function, and wherein the impaired lung function is determined by an increase in lung density in the subject, and the improved lung function is determined by a decrease in lung density in the subject. In another embodiment, the present invention provides a method of treating a radiation induced disorder, wherein the disorder results in impaired lung function, and the method improves the impaired lung function, wherein the impaired lung function is determined by a decrease in fraction potential airspace in the subject, and the improved lung function is determined by an increase in fraction potential airspace in the subject. In yet another embodiment, the present invention provides a method of treating a radiation induced disorder, wherein the disorder results in impaired lung function, and the method improves the impaired lung function, wherein the impaired lung function is determined by a decrease in lung volume in the subject, and the improved lung function is determined by an increase in lung volume in the subject. In yet another embodiment, the present invention provides a method of treating a radiation induced disorder, wherein the disorder results in impaired lung function, and the method improves the impaired lung function, wherein the impaired lung function is determined by a decrease in PaO₂, in the subject, and the improved lung function is determined by an increase in PaO₂ in the subject. In yet another embodiment, the present invention provides a method of treating a radiation induced disorder, wherein the disorder results in impaired lung function, and the method improves the impaired lung function, wherein the impaired lung function is determined by increased lung remodeling, and the improved lung function is determined by decreased or stabilized lung remodeling.

In one aspect, the present invention provides methods for treating radiation-induced pulmonary disorders. As used herein, the terms “radiation-induced lung disorder,” “radiation-induced pulmonary disorder” and “a pulmonary disorder resulting from thoracic exposure to ionizing radiation” are used interchangeably and refer to a lung disorder resulting from thoracic exposure to ionizing radiation. In one embodiment, the radiation-induced pulmonary disorder is radiation pneumonitis. Subjects having a radiation-induced pulmonary disorder typically present with a nonproductive cough, shortness of breath, and low-grade fever. Upon evaluation, subjects are found to have reduced total lung volume, residual volume, and vital capacity, but unrestricted air flow into and out of the lungs. Diagnosis of a radiation-induced pulmonary disorder is based on symptoms including dyspnea, nonproductive cough, and low-grade fever; and generally involves blood tests, e.g., measurement of partial oxygen saturation of the blood; pulmonary function tests, e.g., measurement of total lung volume, residual volume, and vital capacity; and computed tomography (CT) scans of the thorax, e.g., to measure lung density and monitor lung remodeling. As used herein, “thoracic exposure to ionizing radiation” refers to exposure of at least the thorax of the subject to a source of ionizing radiation.

In one aspect, the present invention provides methods and medicaments for treatment of radiation-induced pulmonary disorders. In particular embodiments, the radiation-induced pulmonary disorder is radiation pneumonitis. In various embodiments, the radiation-induced pulmonary disorder is due to thoracic exposure to ionizing radiation. The present invention also provides methods and medicaments for pre-treating an individual that will have or is at increased risk of having thoracic exposure to ionizing radiation, thereby preventing or reducing the severity of a subsequent radiation-induced pulmonary disorder.

In another aspect, the present invention provides medicaments for treatment of a pulmonary disorder resulting from thoracic exposure to ionizing radiation. In one embodiment, the present invention provides the use of an anti-CTGF agent in preparing a medicament for treating a radiation-induced pulmonary disorder, particularly radiation pneumonitis. The medicament may be used to prevent, reduce, reverse, and/or stabilize various pathological features of radiation-induced pulmonary disorders. Such features include, but are not limited to, decreasing lung volume, increasing lung density, remodeling of lung tissue, decreasing PaO₂, and decreasing survival rate. In another embodiment, the present invention provides the use of an anti-CTGF agent in preparing a medicament for preventing or reducing a pulmonary disorder in a subject that will have or is at risk of having thoracic exposure to ionizing radiation. In various embodiments, the medicament also improves lung function in a subject having a radiation-induced pulmonary disorder.

Therefore, in one embodiment, the present invention provides methods for treatment of a pulmonary disorder resulting from thoracic exposure to ionizing radiation, wherein the method comprises administering to the subject in need an anti-CTGF agent. In one embodiment, the present methods can be used to treat a subject having a radiation-induced pulmonary disorder including, but not limited to, radiation pneumonitis. In another aspect, the present invention provides methods for pretreatment of a subject having or at risk of having thoracic exposure to ionizing radiation, wherein the method comprises administering to the subject in need an anti-CTGF agent. The methods of the present invention prevent, reduce, reverse, and/or stabilize various pathological features of radiation-induced pulmonary disorders. Such features include, but are not limited to, decreased lung volume, increased lung density, remodeled lung tissue, decreased PaO₂, and increased mortality. Thus, in one embodiment, the present methods provide a method of reducing, reversing, or stabilizing a pathological feature of a radiation-induced pulmonary disorder in a subject, the method comprising administering to the subject an anti-CTGF agent, thereby reducing, reversing, or stabilizing the pathological feature of the disorder. In another embodiment, the present invention provides a method for pre-treating a subject having or at risk of having thoracic exposure to ionizing radiation to prevent or reduce a resulting pathological feature of radiation-induced pulmonary disorder, the method comprising administering to the subject an anti-CTGF agent, thereby preventing or reducing a resulting pathological feature of the disorder. In various embodiments, the pathological feature of radiation-induced pulmonary disorder is selected from the group consisting of decreased lung volume, increased lung density, remodeled lung tissue, decreased PaO₂, and increased mortality.

In one embodiment, the present invention provides a method of reducing or stabilizing lung density in a subject having increased lung density due to a radiation-induced pulmonary disorder, the method comprising administering to the subject an anti-CTGF agent, thereby reducing or stabilizing lung density in the subject. In another embodiment, the present invention provides methods of preventing or reducing an increase in lung density in a subject that will have or is at risk of having thoracic exposure to ionizing radiation, the method comprising administering to the subject an anti-CTGF agent, thereby preventing or reducing an increase in lung density in the subject. Lung density may be measured by any method known to one of skill in the art. In one particular aspect, the lung density is measured using lung images from computed tomography (CT) scan; more particularly, from high resolution CT (HRCT) scan. In particular embodiments, lung density is measured in Hounsfield Units (HU), and improvement in lung density as a result of the present methods is measured as a decrease in measured HU. Although lung density as measured on the Hounsfield scale vary widely with species (e.g., for humans a normal lung density range is about −800 HU to −900 HU, for mice a normal lung density range is about −400 HU to −500 HU), in general, lung zones with a density between −1,000 HU and −500 HU are typically considered within a normal aerated range, while those between −500 HU and −100 HU are poorly aerated and those between −100 HU and +100 HU are nonaerated. In some embodiments, the subject having increased lung density has a lung density of greater than −500 HU, greater than −400 HU, or greater than −300 HU. In some embodiments, the subject having increased lung density has a lung density between −500 HU and +100 HU, between −500 HU and −100 HU, or between −500 HU and −300 HU. Improvement in lung density may additionally be measured by or associated with improved lung function as described infra.

In another embodiment, the present invention provides a method of reducing or stabilizing lung remodeling in a subject having a radiation-induced pulmonary disorder, the method comprising administering to the subject an anti-CTGF agent, thereby reducing or stabilizing lung remodeling in the subject. In another embodiment, the present invention provides a method of preventing or reducing lung remodeling in a subject that will have or is at risk of having thoracic exposure to ionizing radiation, the method comprising administering to the subject an anti-CTGF agent, thereby preventing or reducing lung remodeling in the subject. Lung remodeling may be measured by any method known to one of skill in the art. In some embodiments, lung remodeling is measured using lung images from CT scan; more particularly, by HRCT. In other embodiments, lung remodeling is measured by lung biopsy and histology. In subjects having radiation-induced pulmonary disorders, portions of normal lung may be replaced by fibrotic septae between dilated airspaces, the gross appearance being referred to as ‘honeycomb changes.’ Lung remodeling in radiation-induced pulmonary disorders generally shows patchy, heterogeneous regions of dense fibrosis and mild or moderate interstitial lymphoplasmacytic infiltrates, architectural remodeling, honeycomb change, and fibroblastic foci. Fibroblastic foci represent zones of disease activity whose extensiveness has been linked to survival. In some embodiments, lung remodeling is measured as a change in fraction potential airspace, i.e., the fraction of lung not occupied by tissue as assessed, e.g., by histology of biopsy material. In particular embodiments, the present methods are used to treat a subject having decreased fraction potential airspace relative to normal. In other embodiments, lung remodeling is measured by percentage of lung showing honeycomb changes or fibroblastic foci. In particular embodiments, the present methods are used to treat a subject having increased percentage of honeycomb change or increased number of fibroblastic foci. Lung remodeling may additionally be measured by or associated with improved lung function as described infra.

Subjects having radiation-induced pulmonary disorders are at high risk of acute or chronic respiratory failure and cardiovascular complications which lead to increased mortality. Thus, in yet another embodiment, the present invention provides a method of increasing the likelihood of survival in a subject having thoracic exposure to ionizing radiation, the method comprising administering to the subject an anti-CTGF agent, thereby increasing the likelihood of survival in the subject. Increased likelihood of survival may also be associated with improved lung function as described infra.

In various embodiments, the methods of the invention improve lung function, in particular embodiments, the methods improve lung function in a subject having impaired lung function resulting from a radiation-induced disorder. Improved lung function may be determined by any measure known to those of skill in the art. In some embodiments, lung function is determined by measuring blood gas parameters, e.g., partial arterial pressure of oxygen (PaO₂) or percent oxygen saturation of blood. For example, improved lung function can be determined in subjects having low PaO₂ by measuring the ability of the present methods to increase PaO₂. Typically, values for PaO₂ greater than about 75-80 mmHg are considered normal, whereas values of 75 mmHg or less indicate a state of hypoxia or hypoxemia. Although oxygen saturation (O₂ Sat) usually correlates with PaO₂, the relationship is not linear. For non-shifted O₂ Sat, a value of 100% corresponds to 90 mmHg PaO₂, a value of 90% corresponds to 60 mmHg, and a value of 60% corresponds to 30 mmHg. Factors that can cause a shift in the correlative values include temperature and pH. In various embodiments, the present methods are used to treat a subject having a PaO₂ of below 80 mmHg, particularly below 75 mmHg, and more particularly below 70 mmHg. In particular embodiments, blood gas parameters are normalized (i.e., return to a level that is at or near the normal level for the particular species for the particular parameter) over the treatment time course.

In some embodiments, lung function is determined by measuring lung volume parameters, e.g., vital capacity, residual volume, and/or total lung volume. Total lung volume or total lung capacity refers to the volume in the lungs upon maximal inspiration, and in a normal adult is 4-6 Liters. Residual volume refers to the volume remaining in the lungs after maximal expiration, and in a normal adult is 1-2.4 Liters. The vital capacity is the maximal volume expelled from the lungs after maximal inspiration. Subjects having radiation-induced pulmonary disorders typically are found to have reduced total lung volume, residual volume, and vital capacity, but unrestricted air flow into and out of the lungs. Thus, the present methods may improve lung function by increasing total lung volume, residual volume, and/or vital capacity in a subject having reduced lung function associated with radiation-induced pulmonary disorders. In certain embodiments wherein the subject has below normal lung vital capacity and/or total lung volume, the methods increase lung vital capacity and/or total lung volume. In particular embodiments, lung volume parameters are normalized over the treatment time course.

In some embodiments, lung function is determined by measuring the cellular make-up of bronchoalveolar lavage (BAL) fluid. At early stages of a radiation-induced pulmonary disorder, the alveolar and adjacent capillary endothelial cells become leaky, leading to alveolar and interstitial edema, and the number of immune cells found in BAL fluid increases. In particular, the number of polymorphonuclear leukocytes (PMNs), which normally comprise about 1-3% of the cellular component of BAL, can increase to 20% or more. Therefore, in various embodiments, the present methods are used to treat a subject having an elevated percentage of PMNs in BAL fluid and the methods of the invention reduce the percentage of PMNs in BAL fluid. In particular embodiments, the present methods are used to treat a subject having greater than 5% PMNs in BAL fluid, particularly greater than 10%, and more particularly greater than 15%. In particular embodiments, the cellular make-up of BAL fluid is normalized over the treatment time course.

In one aspect, the present invention provides methods for treating radiation-induced cardiac disorders. As used herein, the terms “radiation-induced cardiac disorder,” “radiation-induced heart disorder” and “a cardiac disorder resulting from thoracic or mediastinal exposure to ionizing radiation” are used interchangeably and refer to a heart disorder resulting from exposure, typically thoracic or mediastinal exposure, to ionizing radiation. In one embodiment, the radiation-induced heart disorder is radiation pericarditis, radiation-induced constrictive pericarditis, radiation pericardial effusion, or radiation-induced fibrosis. Patients having pericarditis or pericardial effusion may present with dypsnea, chest pain and fever, and can generally be detected with chest x-ray and confirmed by echocardiogram. Constrictive pericarditis can be detected by techniques such as echocardiography, HCRT scanning and magnetic resonance imaging. Acute and subacute forms of pericarditis (which may or may not be symptomatic) may deposit fibrin, which may, in turn, evoke a pericardial effusion. This often leads to pericardial organization, chronic fibrotic scarring, calcification, and restricted cardiac filling. The common features of radiation-induced cardiac complications stem from microcirculation injury with endothelial damage, capillary rupture, and platelet adhesion. This sets up an inflammatory response, which may either resolve or organize to form adhesions between the visceral pericardium and the parietal pericardium, which leads to constriction. Generally, radiation-induced constrictive pericarditis presents 5-10 years after radiation therapy and is more likely to present with an associated pericardial effusion. Although obtaining a careful history and performing a physical examination remain the cornerstones of evaluation, technologic advances have facilitated diagnosis, particularly with the appropriate use of Doppler echocardiography, high-resolution computed tomography (CT), magnetic resonance imaging (MRI), and invasive hemodynamic measurement. Pathological features associated with a radiation-induced heart disorder include pericardial thickening, pericardial effusion, cardiac fibrosis, cardiac remodeling, endothelial damage, pericardial adhesions, and pericardial calcification. As demonstrated herein, the methods of the present invention are effective to treat, prevent, reduce, reverse, or stabilize a radiation-induced heart disorder. As particularly demonstrated herein, application of the method of the invention can prevent, reverse or reduce the development of cardiac fibrosis in a subject having a radiation-induced heart disorder. Therefore, in one embodiment, the present invention provides a method of treating a radiation-induced heart disorder in a subject having, or at risk of having, a radiation-induced heart disorder, the method comprising administering an anti-CTGF agent to the subject, thereby treating the radiation-induced heart disorder. In one aspect the invention provides a method of treating, preventing, reducing, reversing, or stabilizing a radiation-induced heart disorder in a subject having, or at risk of having, a radiation-induced heart disorder, the method comprising administering an anti-CTGF agent to the subject, thereby treating, preventing, reducing, reversing, or stabilizing the radiation-induced heart disorder. In a further aspect, the invention provides a method of treating, preventing, reducing, reversing, or stabilizing a pathological feature associated with a radiation-induced heart disorder in a subject having, or at risk of having, a radiation-induced heart disorder, the method comprising administering an anti-CTGF agent to the subject, thereby treating, preventing, reducing, reversing, or stabilizing the pathological feature. The treating, preventing, reducing, reversing, or stabilizing of the pathological feature can be determined by any method known in the medical art including those described above for detecting a radiation-induced heart disorder, e.g., Doppler echocardiography, high-resolution computed tomography (CT), magnetic resonance imaging (MRI), and invasive hemodynamic measurement. Any competent medical practicioner can readily determine the appropriate pathological feature(s) to monitor to determine efficacy of the method.

The methods of the present invention, as exemplified herein, clearly demonstrate that subjects show significant improvement in lung density, lung remodeling, lung function, and survival in all treatment periods. Thus, the methods provide significant benefit in a subject whether the methods are initiated prior to exposure to ionizing radiation or at any time subsequent to exposure to ionizing radiation. The data demonstrate that, to the extent possible, the methods should be initiated as early as possible and maintained throughout the period that the subject remains at risk for diminished lung function and compromised survivability. The data also demonstrate that patients in the chronic progressive phase of the disease still benefit from the methods and medicaments of the present invention, improving lung function, reversing lung remodeling, and reducing mortality.

Therefore, in some embodiments, the methods of the present invention are initiated upon diagnosis of a radiation-induced disorder. In various embodiments, the methods may be initiated prior to an event associated with increased probability or likelihood of being exposed to ionizing radiation. In one embodiment, the subject is at increased risk of exposure to ionizing radiation due to an occupational event. For example, the methods of the present invention may be used to pre-treat a worker that may be exposed to ionizing radiation as part of a mining project. In another embodiment, the subject is at increased risk of exposure to ionizing radiation due to a medical event, e.g., exposure to a nuclear medical therapy or procedure known or suspected of causing radiation-induced disorders. For example, the methods of the present invention may be used to pre-treat a patient that is going to be exposed to ionizing radiation as part of radiation therapy.

### Subjects

In some embodiments, the subject suitable for, or in need of, treatment with the methods and anti-CTGF agents of the present invention is an individual, preferably a mammal, more preferably a human, who has, or is at risk of having, a radiation-induced disorder, typically resulting from ionizing radiation exposure. In particular embodiments, the subject has been, or will be, exposed to ionizing radiation, typically as a consequence of radiation therapy. Suitable subjects having a radiation-induced disorder can be readily identified by any competent medical practicioner. For example, individuals exhibiting any of the symptoms associated with radiation-induced disorders as are well-known and are described herein, particularly individuals who have been exposed to ionizing radiation several months to several years prior to onset of symptoms. Suitable subjects at risk of having a radiation-induced disorder are individuals who are likely to be exposed to ionizing radiation. Such at risk individuals include those individuals who are probable or likely to be exposed to ionizing radiation due to occupational or environmental risks, for example, in mining operations, nuclear power plants, and long-distance airline travel. Other suitable subjects at risk of having a radiation-induced disorder are individuals who have recently begun or are planning to begin a course of radiation therapy.

In some embodiments, the subject in need of treatment is an individual, preferably a mammal, more preferably a human, who has a radiation-induced lung disorder. In particular embodiments, the radiation-induced lung disorder is radiation pneumonitis. In other embodiments, the subject in need of treatment is an individual, preferably a mammal, more preferably a human, who has a radiation-induced heart disorder. In particular embodiments, the radiation-induced lung disorder is radiation pericarditis, radiation-induced cardiac effusion, or radiation-induced cardiac fibrosis. In other embodiments, the subject is an individual, preferably a mammal, more preferably a human, who is at increased probability or likelihood of having thoracic exposure to ionizing radiation. In some embodiments, the subject is an individual, preferably a mammal, more preferably a human, who will have or is at increased risk of having thoracic exposure to ionizing radiation. Thoracic exposure to ionizing radiation may occur as an environmental or occupational exposure, such as in mining operations, nuclear power plants, and long-distance airline travel. Thoracic exposure to ionizing radiation may occur as a medical treatment, such as with radiation therapy for lung or breast cancer. In some embodiments, the subject's probability or likelihood of being exposed to ionizing radiation is due to occupational or environmental risks. In other embodiments, the subject's probability or likelihood of being exposed to ionizing radiation is due to therapeutic exposure to ionizing radiation. Such exposure may be due to, e.g., radiation therapy or nuclear medicine treatments. A subject at increased risk of having thoracic exposure to ionizing radiation is an individual that, due to occupational or environmental factors, is more likely to be exposed to radiation. For example, airline personnel who frequently fly long-distances are more frequently exposed to radiation and thus are at increased risk of thoracic exposure to ionizing radiation.

### Anti-CTGF Agent

The methods of the invention are accomplished by administering to a subject in need an anti-CTGF agent. As used herein, the terms “anti-connective tissue growth factor agent,” or “anti-CTGF agent,” refer to any agent, molecule, macromolecule, compound, or composition that specifically and directly inhibits or reduces the activity or function of the CTGF protein, or specifically and directly inhibits or reduces the expression of the CTGF gene. The anti-CTGF agent is one that is specific for CTGF and exerts its effect directly and specifically on the CTGF protein or on the CTGF gene or mRNA, rather than a non-specific inhibitor (e.g., a non-specific protease or transcription inhibitor) or an indirect inhibitor (e.g., an inhibitor of a component of an upstream or downstream signaling pathway for CTGF). As used herein, “connective tissue growth factor” and “CTGF” refer to a matricellular protein belonging to a family of proteins identified as CCN proteins. CTGF may also be referred to within the art as “hypertrophic chondrocyte-specific protein 24,” “insulin-like growth factor-binding protein,” and “CCN2.” Preferred anti-CTGF agents include anti-CTGF antibodies, particularly monoclonal antibodies, and polynucleotide inhibitors of CTGF, particularly anti-CTGF siRNAs, anti-CTGF shRNAs, anti-CTGF miRNAs, and anti-CTGF antisense oligonucleotides.

In some embodiments, polynucleotide inhibitors of CTGF, including small interfering ribonucleic acids (siRNAs), micro-RNAs (miRNAs), and CTGF antisense sequences may be used in the present methods to inhibit expression and/or production of CTGF. (See, e.g., Kondo et al. (2000) Biochem Biophys Res Commun 278:119-124.) Such techniques are well-known to those of skill in the relevant art. Polynucleotide inhibitors that target CTGF expression have been described and utilized to reduce CTGF expression in various cell types. (See, e.g., International Publication No. WO 96/38172; International Publication No. WO 00/27868; International Publication No. WO 00/35936; International Publication No. WO 03/053340; Kothapalli et al. (1997) Cell Growth Differ 8:61-68; Shimo et al. (1998) J Biochem (Tokyo) 124:130-140; Uchio et al. (2004) Wound Repair Regen 12:60-66; Guha et al. (2007) FASEB J 21:3355-3368; U.S. Pat. No. 6,358,741; U.S. Pat. No. 6,965,025; U.S. Pat. No. 7,462,602; U.S. Patent Application Publication No. 2008/0070856; U.S. Patent Application Publication No. 2008/0176964.) CTGF antisense constructs and other types of polynucleotide inhibitors of CTGF can be used to inhibit or reduce expression of CTGF and thereby treat radiation-induced disorders. Such constructs can be designed using appropriate vectors and expressional regulators for cell- or tissue-specific expression and constitutive or inducible expression. Such genetic constructs can be formulated and administered according to established procedures within the art. The polynucleotide inhibitors used in the present methods and medicaments may be made using solid phase synthesis techniques known to those of skill in the art and available through various vendors including Applied Biosystems (Foster City Calif.). Any other means for such synthesis known in the art may additionally or alternatively be employed.

In some embodiments, the anti-CTGF agent can be an antibody that binds specifically to CTGF. The anti-CTGF antibody may be specific for CTGF endogenous to the species of the subject to be treated or may be cross-reactive with CTGF from a number of species. In some embodiments, the antibody for use in the present methods is obtained from the same species as the subject in need. In other embodiments, the antibody is a chimeric antibody wherein the constant domains are obtained from the same species as the subject in need and the variable domains are obtained from another species. For example, in treating a human subject the antibody for use in the present methods may be a chimeric antibody having constant domains that are human in origin and variable domains that are mouse in origin. In preferred embodiments, the antibody for use in the present methods binds specifically to the CTGF endogenous to the species of the subject in need. Thus, in certain embodiments, the antibody is a human or humanized antibody, particularly a monoclonal antibody, that specifically binds human CTGF (GenBank Accession No. NP_{—}001892.1). In particular embodiments, the antibody is the antibody described and claimed in Lin et al., United States Patent Application Publication No. 2009/0017043, or in U.S. Pat. No. 7,405,274. In some embodiments, the antibody has the amino acid sequence of the antibody produced by the cell line identified by ATCC Accession No. PTA-6006. In other embodiments, the antibody binds to CTGF competitively with an antibody produced by ATCC Accession No. PTA-6006. A particular antibody for use in the present methods is CLN1 or mAb1 as described in U.S. Pat. No. 7,405,274, or an antibody substantially equivalent thereto or derived therefrom. An antibody for use in the present methods may also be a functional fragment such as a Fab, F(ab)2, Fv, or single chain variable fragment (scFV) of any antibody described above. A functional fragment of an antibody will be a fragment with similar (not necessarily identical) specificity and affinity to the antibody which it is derived. An antibody for use in the present methods may also be derived from any antibody described above. Such derivatives may include any suitable antibody derivation known to those of skill in the art and include, but are not limited to, diabodies, triabodies, and minibodies.

As referred to herein, the phrase “an antibody that specifically binds to CTGF” includes any antibody that binds to CTGF with high affinity. Affinity can be calculated from the following equation:

Affinity=Kₐ=[Ab·Ag]/[Ab][Ag]=1/K_{d}

where [Ab] is the concentration of the free antigen binding site on the antibody, [Ag] is the concentration of the free antigen, [Ab·Ag] is the concentration of occupied antigen binding sites, Kₐ is the association constant of the complex of antigen with antigen binding site, and K_{d} is the dissociation constant of the complex. A high-affinity antibody typically has an affinity at least on the order of 10⁸ to 10⁹ M⁻¹. In particular embodiments, an antibody for use in the present methods will have a binding affinity for CTGF on the order of 10⁸ M⁻¹, more particularly on the order of 10⁹ M⁻¹, and more particularly on the order of 10¹⁰ M⁻¹.

### Administration and Formulation of Anti-CTGF Agents

The anti-CTGF agents used in the method of the present invention can be delivered directly or in pharmaceutical compositions containing excipients, as is well known in the art. The anti-CTGF agent can be used in the manufacture of a medicament for treating a radiation-induced disorder. An effective amount of anti-CTGF agent can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Goodman and Gilman's The Pharmacological Basis of Therapeutics, 10^{th} Ed. (2001), Hardman, Limbird, and Gilman, eds. MacGraw Hill Intl.) Typically, the anti-CTGF agent is administered in an amount sufficient to provide therapeutic efficacy over the treatment time course. Therapeutic efficacy can be measured using any parameter provided herein, or as well-known in the medical arts for efficacy in radiation-induced disorders, including improvement in any pathological feature of radiation-induced disorder, such as a radiation-induced lung disorder, and/or improvement in lung function.

In various embodiments, the anti-CTGF agent may be administered at appropriate intervals to achieve the claimed result, such as improved lung function as measured by any of the parameters provided herein. In particular embodiments, the anti-CTGF agent may be administered 1, 2, 3, 4, or 5 or more times per month. In some embodiments, the anti-CTGF agent may be administered 1 time per week or 1 time every other week. In various embodiments, the administration of the anti-CTGF agent is continued until the pathological feature or functional parameter is essentially normalized or the subject is no longer considered at risk. In various embodiments, the anti-CTGF agent may be administered beginning prior to, simultaneous with, and/or subsequent to, exposure of the subject to ionizing radiation. The anti-CTGF agent may be administered to a subject beginning subsequent to ionizing radiation exposure but prior to the manifestation of a radiation-induced disorder. The anti-CTGF agent may be administered to a subject beginning subsequent to ionizing radiation exposure but prior to the manifestation of symptoms of the chronic phase of a radiation-induced disorder. The anti-CTGF agent may be administered to a subject beginning subsequent to the manifestation of symptoms of acute or chronic phases of a radiation-induced disorder. The anti-CTGF agent is preferably administered to a subject prior or subsequent to ionizing radiation exposure but before the manifestation of a radiation-induced disorder. Typically, particularly in embodiments for treatment of radiation-induced disorders resulting as a consequence of radiation therapy, the anti-CTGF agent will be administered beginning shortly before (e.g., several days to several hours before) or shortly after (e.g., several days to several hours after) the exposure of the subject to the radiation, and will continue for several weeks to several months, or throughout the expected acute response phase of the radiation-induced disorder.

In various embodiments, the anti-CTGF agent is administered at appropriate levels to achieve the desired pharmacological effect. In particular embodiments, the anti-CTGF agent is an antibody that binds specifically to CTGF. In various embodiments, the antibody may be administered at a dose of from 0.01 to 100 mg of antibody/kg of patient weight, more particularly from 0.1 to 50 mg/kg, and even more particularly from 1-15 mg/kg. Doses particularly contemplated for use in the present methods include, but are not limited to, 3 mg/kg; 5 mg/kg; 10 mg/kg; and 12 mg/kg. Depending on the type and severity of the disease, about 0.015 to 15 mg/kg is an initial candidate dosage for administration to the patient, whether, for example, by one or more separate administrations, or by continuous infusion. For repeated administrations over several days or longer, depending on the condition, the treatment is repeated until a desired suppression of disease symptoms occurs. However, other dosage regimens may be useful and are not excluded from the present invention.

In various embodiments, the anti-CTGF agent may be administered by any route that provides a suitable pharmacokinetic profile. In particular embodiments, the anti-CTGF agent is administered intravenously. In some embodiments, the anti-CTGF agent is administered intravenously in a single bolus injection, or by continuous infusion over a period of time, and/or by intramuscular, subcutaneous, intra-articular, intrasynovial, intrathecal, intravitreal, intracranial, oral, topical, or inhalation routes. In other embodiments, the anti-CTGF agent is administered intravenously by infusion. In other embodiments, the anti-CTGF agent may be administered subcutaneously, intramuscularly, or intraperitoneally. When the anti-CTGF agent possesses the suitable activity, intratumoral, peritumoral, intralesional, or perilesional routes of administration can also be utilized to exert local as well as systemic therapeutic effects.

When the methods of the invention are used prior to exposure to ionizing radiation, the methods may additionally decrease the sensitivity of normal tissue to ionizing radiation. As provided herein, pretreatment using the methods of the invention prior to thoracic exposure to ionizing radiation improved lung density and lung function, and improved survival rates in subjects subsequent to exposure. The invention therefore contemplates that when the methods of the invention are applied prior to exposure, the subject could receive a higher level of radiation exposure without significant loss of lung function. This is particularly useful in radiation therapy, where the methods of the invention can be used to pre-treat a subject prior to thoracic exposure to ionizing radiation, thereby allowing higher doses of radiation and/or more frequent dosing of radiation without adversely affecting lung function and survivability of the subject. The ability to apply higher doses of radiation or more frequent dosing would improve the likelihood of eradicating the tumor.

As described elsewhere herein, any anti-CTGF agent that directly and specifically inhibits the expression or activity of CTGF may be used in formulating the present medicaments. In particular embodiments, the anti-CTGF agent is an antibody that binds specifically to CTGF, or a polynucleotide inhibitor of CTGF expression (for example, an antisense oligonucleotide, siRNA, shRNA, or miRNA). In a preferred embodiment, the anti-CTGF agent is an antibody that binds specifically to CTGF. Any antibody that specifically binds to CTGF may be used in formulating the present medicaments. Antibodies for use in the present medicaments are described supra. In various embodiments, the antibody for use in the present medicaments is an antibody described and claimed in Lin et al., United States Patent Application 2009/0017043, or in U.S. Pat. No. 7,405,274. In particular embodiments, the antibody has the amino acid sequence of the antibody produced by the cell line identified by ATCC Accession No. PTA-6006. In other embodiments, the antibody competitively binds to CTGF with an antibody produced by ATCC Accession No. PTA-6006. A particular antibody for use in the present medicaments is CLN1 or mAb1 as described in U.S. Pat. No. 7,405,274, or an antibody substantially equivalent thereto or derived therefrom.

The medicament may be formulated for the intended route of administration. Such formulations may encompass pharmaceutically acceptable carriers that are inherently nontoxic and nontherapeutic. Polynucleotide inhibitors may be admixed, encapsulated, conjugated or otherwise associated with other molecules, molecule structures, or mixtures of compounds, e.g., liposomes, receptor targeted molecules, oral, rectal, topical or other formulations, for assisting in uptake, distribution and/or absorption. (See, e.g., U.S. Pat. No. 5,108,921; U.S. Pat. No. 5,354,844; U.S. Pat. No. 5,416,016; U.S. Pat. No. 5,459,127; U.S. Pat. No. 5,521,291; U.S. Pat. No. 5,543,158; U.S. Pat. No. 5,547,932; U.S. Pat. No. 5,583,020; U.S. Pat. No. 5,591,721; U.S. Pat. No. 4,426,330; U.S. Pat. No. 4,534,899; U.S. Pat. No. 5,013,556; U.S. Pat. No. 5,108,921; U.S. Pat. No. 5,213,804; U.S. Pat. No. 5,227,170; U.S. Pat. No. 5,264,221; U.S. Pat. No. 5,356,633; U.S. Pat. No. 5,395,619; U.S. Pat. No. 5,416,016; U.S. Pat. No. 5,417,978; U.S. Pat. No. 5,462,854; U.S. Pat. No. 5,469,854; U.S. Pat. No. 5,512,295; U.S. Pat. No. 5,527,528; U.S. Pat. No. 5,534,259; U.S. Pat. No. 5,543,152; U.S. Pat. No. 5,556,948; U.S. Pat. No. 5,580,575; and U.S. Pat. No. 5,595,756.) Examples of carriers for use with antibodies include ion exchangers, alumina, aluminum stearate, lecithin; serum proteins such as human serum albumin; buffers such as phosphate, histidine, or glycine; sorbic acid, potassium sorbate, partial glyceride mixtures of saturated vegetable fatty acids, water, salts; or electrolytes such as protamine sulfate, sodium chloride, metal salts, colloidal silica, magnesium trisilicate, polyvinyl pyrrolidone, cellulosic polymers, and polyethylene glycol. Carriers for topical or gel-based forms of antibody include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. Conventional depot forms include, for example, microcapsules, nano-capsules, liposomes, plasters, sublingual tablets, and polymer matrices such as polylactide:polyglycolide copolymers. When present in an aqueous dosage form, rather than being lyophilized, the antibody typically will be formulated at a concentration of about 0.1 mg/ml to 200 mg/ml, although wide variation outside of these ranges is permitted.

The medicaments may, if desired, be presented in a pack or dispenser device containing one or more unit dosage forms containing the active ingredient. Such a pack or device may, for example, comprise metal or plastic foil, such as a blister pack; or glass and rubber stoppers such as in vials. The pack or dispenser device may be accompanied by instructions for administration. Compositions comprising an agent of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention will be further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Materials and Methods

Experimental Protocol and Animal Model Female C57BL/6 mice (8-wk-old; approximate body weight: 20 g; Charles River Laboratories) were randomized into 8 groups of 25 animals and were supplied with food and water ad libitum. Six groups (150 mice) were irradiated (IR) with a single dose of 20 Gy at day 0. Mice were anesthetized by intraperitoneal application of 0.2 mg/kg Rompun (Bayer) and 100 mg/kg ketamin 10% (Parke-Davis). Photon irradiation was administered as a single 20 Gy dose to the entire thorax (Siemens linear accelerator; source surface distance of 1 m; irradiation field of 0.02×0.2 m). Other organs, above and below the thorax, were shielded.

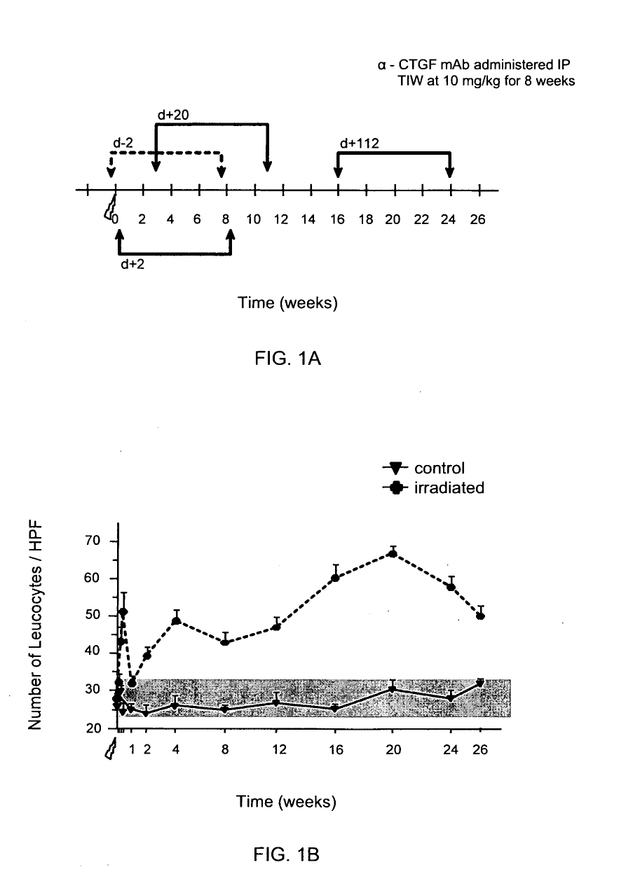

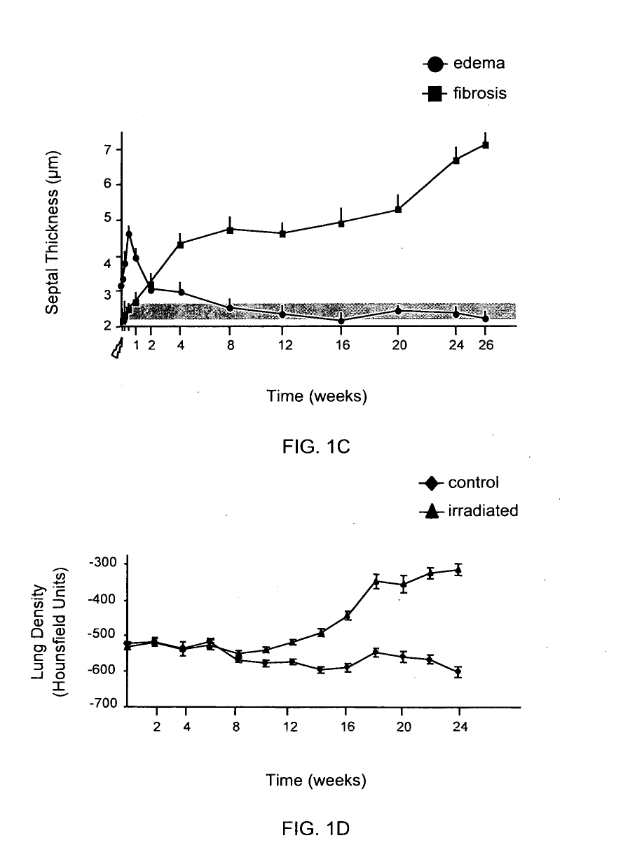

The anti-CTGF monoclonal antibody (αCTGF mAb) used in this experiment is described and claimed in U.S. Pat. No. 7,405,274. Irradiated mice were administered either no therapeutic (25 mice), control IgG (25 mice), or αCTGF mAb (100 mice). Animals receiving αCTGF mAb were randomized into groups to receive antibody therapy beginning either 2 days before (25 mice; d−2), 2 days after (25 mice; d+2), 20 days after (25 mice; d+20), or 112 days after (25 mice; d+112) irradiation. In each αCTGF mAb group, antibody was administered at a dose of 10 mg/kg by intraperitoneal (i.p.) injection three times per week for a dosing period of 8 weeks. The two nonirradiated groups received either control IgG or αCTGF mAb for the first 8 weeks of the experiment. A schematic of the different dosing schedules is provided in FIG. 1A.

### Lung Imaging

To obtain independent qualitative and quantitative measures for lung remodeling in the mice, high resolution computed tomography (HRCT) was performed in every surviving mouse from each group on weeks 4, 8, 12, 16, 18, 20, 22, 24, 30 and 48 after irradiation. CT images were captured on a SOMATOM PLUS 4 VOLUME ZOOM multi-slice CT scanner (Siemens). 120 kV with 100 mAS were applied. 0.5-mm thin slices with 0.5-mm inter-slice distance spanned the complete mouse chest (a total acquisition time of 0.5 s). Multiplanar reconstructions were performed for semiquantitative analysis. The Hounsfield units (HU) of section slices from the upper, middle and lower lung regions were determined. Density on CT is often described by Hounsfield Units, where pure water measures 0 HU, air measures −1,000 HU, and very dense structures such as bone approach +1,000 HU. After an initial examination of the whole lung, representative slides were chosen to undergo further analysis. Three slides of the lung, representing the upper (5 slides below the apex), middle (divorce of the trachea) and lower (about 5 slides above the diaphragmatic dome) region were selected and measured quantitatively by Hounsfield Units. Six circles were set in the selected fields of both sides of the lung, representing the upper anterior and posterior, the middle anterior and posterior and the lower anterior and posterior region, thus collecting twelve sets of data per mouse. Circles were set as large as possible, but avoiding big bronchi and vessels. All examinations were performed with the same window and level settings (400/1000). Total arithmetic means±standard error of the mean (SEM) of the HU were calculated.

### Lung Histology

Histological analysis from mice tissues was performed as described in Plathow et al. (2004) Invest. Radiol. 39:600-609. In brief, lungs were fixed by intratracheal instillation of 4% formalin followed by overnight fixation, embedded in paraffin, sectioned at 5 μm, and stained with hematoxylin and eosin (H&E), Sirius red or Masson's trichrome. The total count of leukocytes, and septal thickness were determined by morphometric evaluation (Q 600 Quantimet; Leica).

### Statistics

Mouse survival curves after thoracic irradiation and αCTGF mAb treatments were calculated with the Kaplan-Meier method and compared using the log-rank test. Other quantitative data are given as mean values±SEM or as indicated. For analysis of differences between the groups, ANOVA followed by the appropriate post hoc test for individual comparisons between the groups was performed. All tests were two-tailed. P<0.05 was considered statistically significant.

### Results

### Course of Radiation-Induced Lung Damage

The present model of radiation-induced pulmonary disorders utilizes a single dose of ionizing radiation as an initial insult. Exposure of normal lung tissue to irradiation produces an acute pneumonitis and a progressive, long-term fibrosis (see, e.g., Movsas et al. (1997) Chest 111:1061-1076). Characteristic histologic findings in the pneumonitis phase of the radiation response include prominent inflammatory cell infiltrates in the alveoli and lung interstitium with simultaneous interstitial edema. Both parameters typically exhibit similar kinetics in the acute phase, reaching their maximum about 72 h after radiation injury. After the acute radiation response, both leukocyte count and septal edema spontaneously subside within a few days. (See FIGS. 1B and 1C.)

The later fibrogenesis phase is accompanied by a strong second onset of leukocyte infiltration that typically begins several weeks after irradiation and reaches a peak at about 20 weeks after irradiation. Development of fibrosis by progressive collagen deposition detectable by Masson's trichrome staining of irradiated lungs is usually evident after week 12. This fibrogenesis phase is characterized by development of typical fibroblast foci, with abnormal wound healing/repair leading to replication of mesenchymal cells, as characterized by fibroblast/myofibroblast migration and proliferation and exuberant deposition of extracellular matrix in irradiated lungs. At later time points (>20 wk), the fibroblast foci evolve and coalesce into more widespread fibrosis with remodeling of the lung architecture. The second onset of progressive fibrogenesis-related infiltration of leukocytes typically persists until the morphologically described fibrosis process is completed (after week 26). (See FIGS. 1B and 1C.) Fibrosis can be quantitatively assessed by measuring lung density (quantified in Hounsfield units, HU) using HRCT. Lung density dramatically increases during weeks 12-24 after radiotherapy in irradiated animals. (See FIG. 1D.)

### Lung Remodeling Shown by Histology

To monitor the pathogenesis of the radiation-induced lung remodeling process and to evaluate the effects of αCTGF mAb, mice were selected for analysis of leukocyte infiltration and collagen deposition with associated thickening of the alveolar septum. Histological examination of H&E stained lung sections taken from mice at various timepoints after irradiation demonstrated that significant lung remodeling occurred between 13 and 19 weeks after irradiation in the irradiated but untreated mice, and administration of αCTGF mAb attenuated this remodeling in a schedule-dependent manner in the mice treated with αCTGF mAb (data not shown).

### Potential Airspace

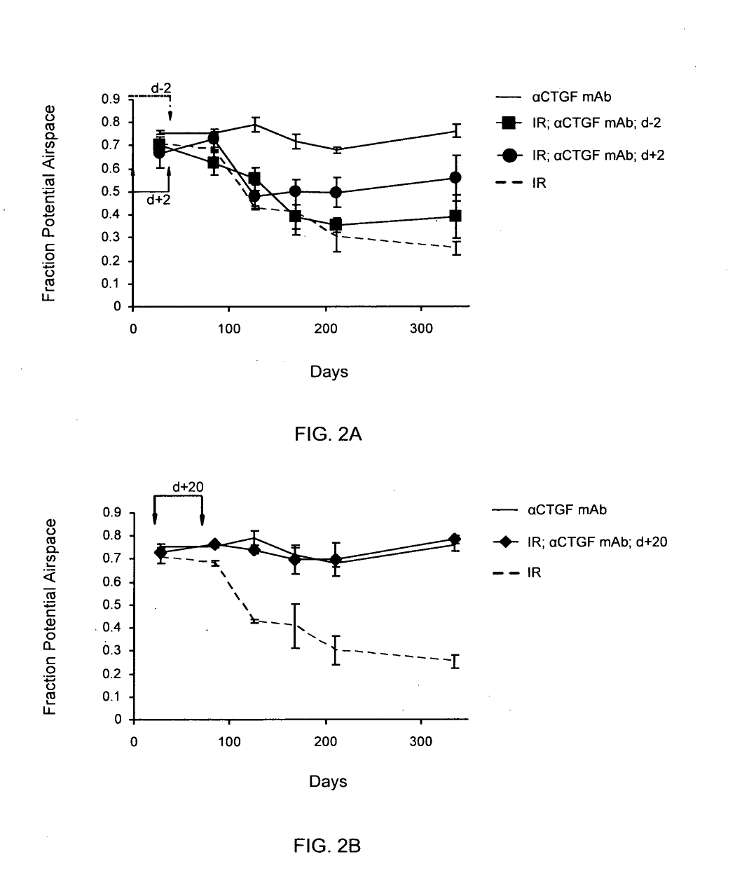

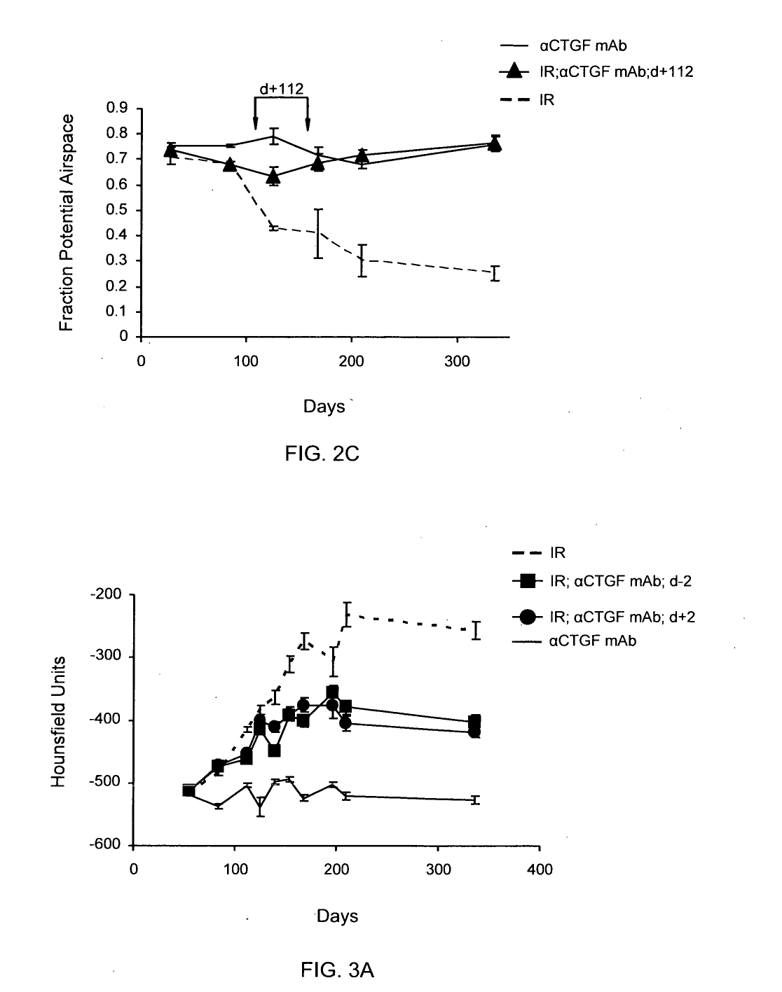

Evaluation of potential airspace in the lung, as assessed by image analysis of histological specimens, demonstrated that the present methods attenuate lung remodeling. (FIG. 2) Image analysis of the H&E stained lung sections from the various groups were processed to quantify the amount of solid tissue vs. empty space. The ratio of empty space to tissue was called the fraction potential airspace. No significant change in potential airspace was observed in the first 12-13 weeks after irradiation in any of the groups. By week 19, however, the fraction potential airspace in the irradiated but untreated mice significantly decreased and continued to decrease until week 31. By week 31 after irradiation, all of the groups that had been treated with αCTGF mAb exhibited larger fraction potential airspace than the irradiated untreated group. As shown in FIG. 2A, potential airspace in irradiated lungs improved when αCTGF mAb administration was initiated 2 days before or 2 days after irradiation and continued for 8 weeks. As shown in FIGS. 2B and 2C, potential airspace in irradiated lungs was essentially normalized (that is, was very similar to the potential airspace seen in unirradiated mice) when αCTGF mAb administration was initiated 20 days or even 112 days after irradiation, and treatment was continued for 8 weeks. Thus, the methods of the present invention attenuate lung remodeling in a subject having a radiation-induced pulmonary disorder.

### Lung Density

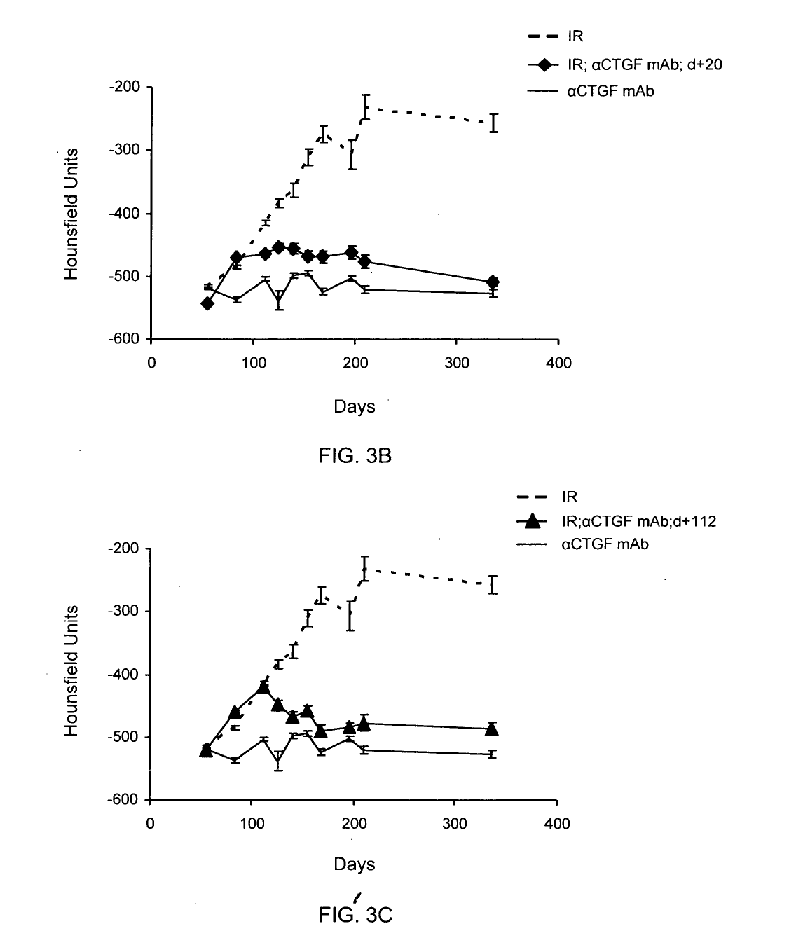

To monitor changes in lung density following irradiation and to evaluate the effects of αCTGF mAb on the changes in lung density, the density of the lungs of all mice were measured by micro-CT at various times after irradiation. The density of the lungs of unirradiated mice (treated with either IgG or αCTGF mAb) was unchanged over the course of the experiment. In contrast, the lung densities in irradiated but untreated mice (either no treatment or treatment with IgG) progressively increased until about 30 weeks after irradiation, after which, the lung densities of the few surviving mice did not increase further. The changes in lung density observed in the irradiated but untreated mice were attenuated by the present methods when αCTGF mAb was administered for 8 weeks beginning immediately before (d−2) or after (d+2) irradiation (FIG. 3A.) Administration of αCTGF mAb beginning 20 days (d+20) or 112 days (d+112) after irradiation had a more dramatic effect on lung densities, such that the lung densities in the irradiated groups treated using the methods of the present invention were indistinguishable from those of nonirradiated mice (FIGS. 3B and 3C.) Further, administration of αCTGF mAb 112 days after irradiation showed a reversal of lung density increases that occurred prior to treatment initiation (FIG. 3C.) Together, these data demonstrate that the methods of the invention are capable of reducing, preventing, stabilizing, and reversing the increased lung density that occurs in a subject having a radiation-induced pulmonary disorder.

### Partial Pressure O₂ and Oxygen Saturation Percent

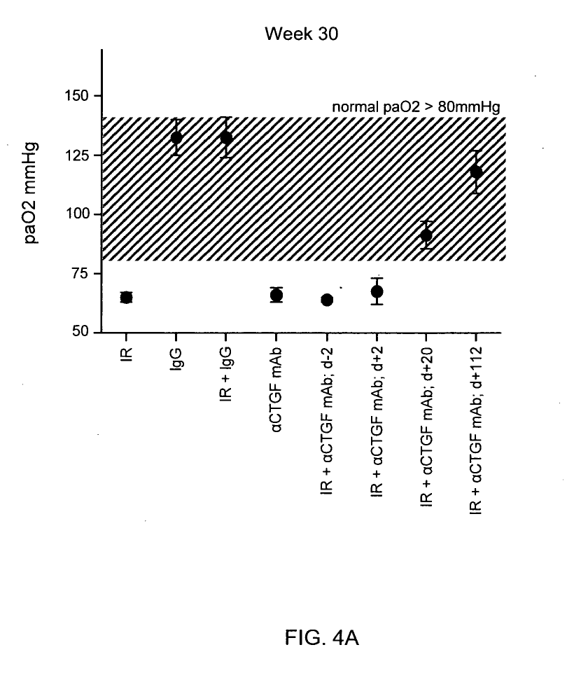

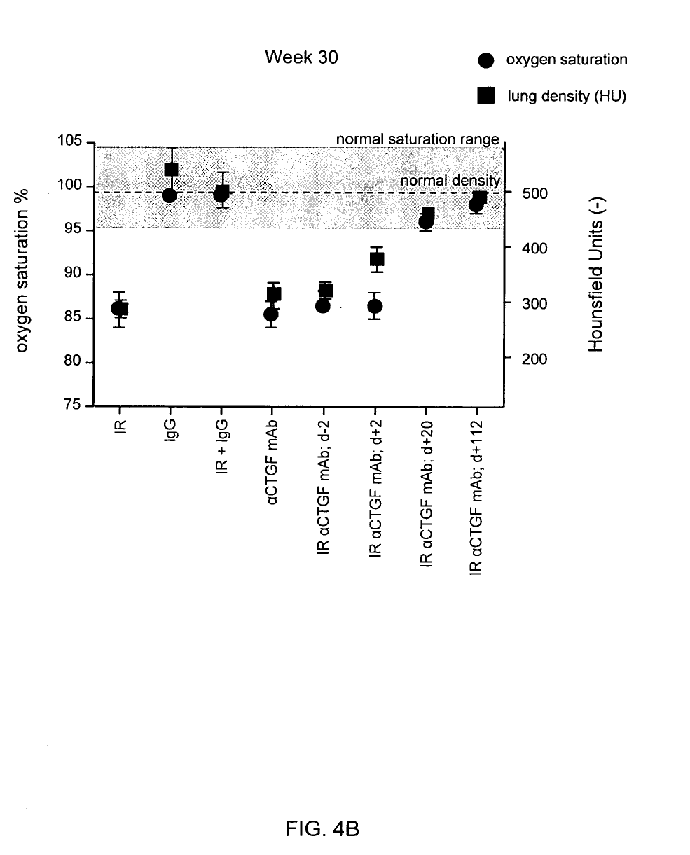

To monitor changes in lung function and to evaluate the effects of αCTGF mAb, blood was sampled from the tail capillary of mice in various groups and oxygen partial pressure was measured. FIG. 4 sets forth blood partial oxygen pressure (PaO₂) 30 weeks after irradiation for the treatment groups. The PaO₂ for mice in the unirradiated groups (IgG only or αCTGF mAb only) was in the normal range (normal range is >80 mm Hg shown as the striped area). As can be seen in FIG. 4A, the irradiated but untreated mice (either no treatment or IgG treated) exhibited PaO₂ well below normal. However, irradiated mice that were treated with αCTGF mAb beginning either at 20 days (d+20) or 112 days (d+112) after irradiation had PaO₂ in the normal range (FIG. 4A.) The oxygen partial pressure of the blood was converted to a percent saturation and compared to the lung densities measured at week 30 (FIG. 4B). As can be seen in FIG. 4B, the correlation of the lung density and the oxygen saturation is quite striking and suggested that reduction in lung density is a good surrogate for improvement in lung function, and both parameters are normalized in animals treated with the αCTGF mAb. Additional blood samples were collected at week 48 and measured for PaO₂ (data not shown). No mice remained alive in the irradiated untreated group at this time point but samples from 1-3 mice from the other groups were examined including remaining mice from the irradiated IgG treated group. Oxygen saturation was below normal in the irradiated IgG treated mice, but oxygen saturation was now in the normal range for all of the αCTGF mAb treated groups.

### Septal Thickness

Another way to assess remodeling of the lungs is to measure the thickness of the septa between the alveoli. The thickness of the alveolar septa were measured manually from photographs of lung sections from mice in all groups. The mean of 100 measurements per lung section were plotted as a function of time (data not shown). Little change in the alveolar septa thickness was observed in the unirradiated mice. In contrast, the septa of irradiated and untreated (either no treatment or IgG treated) mice exhibited progressive thickening between 12 and 30 weeks after irradiation. Septa of irradiated mice that were treated with αCTGF mAb beginning 2 days before or 2 days after irradiation also exhibited progressive thickening albeit with different time courses than the irradiated untreated controls. However, septa of irradiated mice that were treated with αCTGF mAb beginning at 20 days or 112 days after irradiation exhibited little change in alveolar septa thickness (although there appeared to be a slight thickening at 12 weeks that subsequently resolved) and at 30 weeks after irradiation were indistinguishable from the unirradiated controls.

### Survival

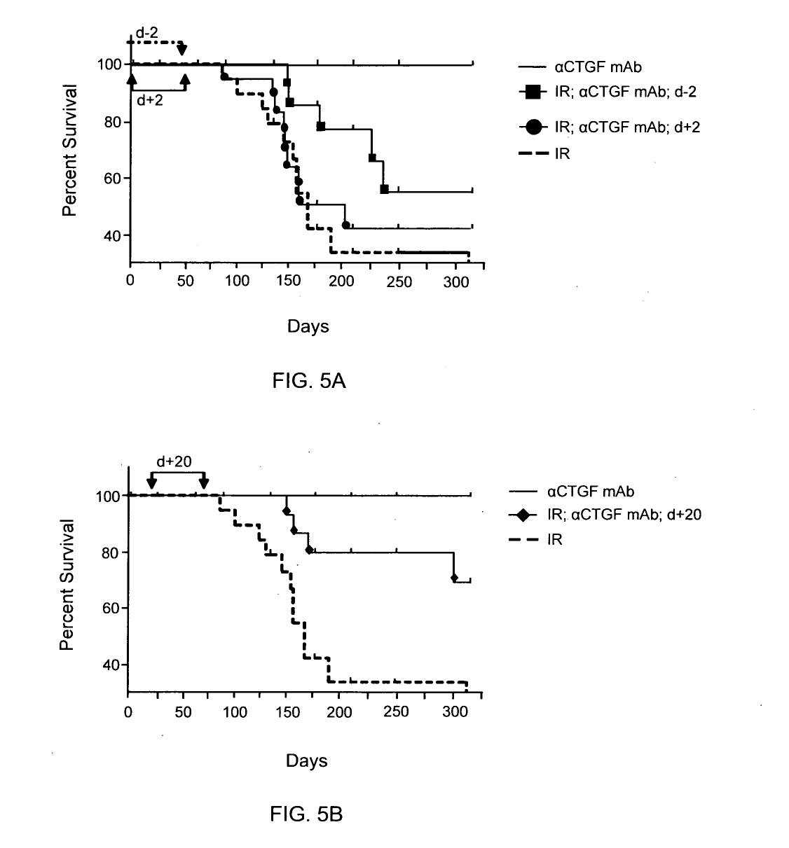

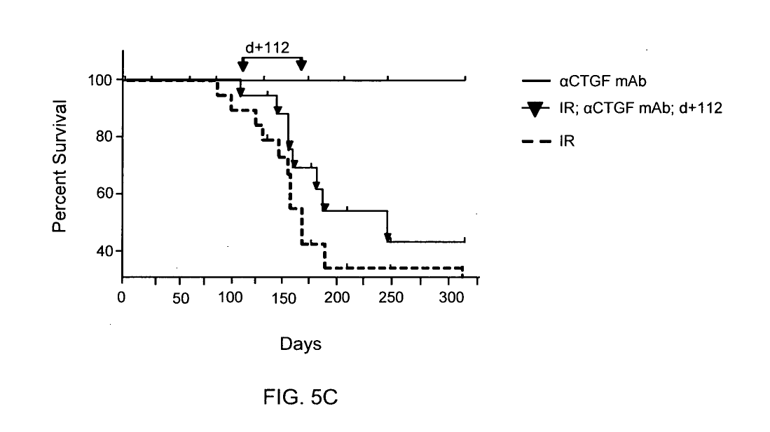

FIG. 5 shows the percent survival of mice from treatment groups over the 48 weeks of the experiment. Over the course of the 48 week experiment, no mice in the unirradiated +αCTGF mAb control group died, while one mouse in the unirradiated+IgG control group died (unirradiated+IgG group is not shown on FIG. 5). The survival of the irradiated and untreated mice (IR) and those that were irradiated and exposed to placebo (IR+IgG) were similar, with a median survival of 167 and 161 days, respectively (irradiated+IgG group not shown on FIG. 5). The group that began receiving αCTGF mAb two days before irradiation (d−2) exhibited a statistically significant improvement in survival (p=0.041) with a median survival>336 days (FIG. 5A). The group that began receiving αCTGF mAb 20 days after irradiation (d+20) also exhibited a statistically significant improvement in survival relative to the irradiated placebo-treated group (p=0.021) with a median survival >336 days FIG. 5B). The group that began receiving αCTGF mAb at 16 weeks after irradiation (d+112) exhibited an increase in median survival (246 days) over the irradiated untreated groups but the increase was not statistically significant (FIG. 5C). The group that began receiving αCTGF mAb 2 days after irradiation (d+2) exhibited a median survival of 202 days (FIG. 5A). Thus all of the irradiated groups that received αCTGF mAb treatment exhibited greater median survival rates after receiving a lethal ionizing radiation dose than the irradiated and untreated (or placebo treated) control groups. Together, these data demonstrate that the methods of the invention improve survivability and survival rate in a subject having a radiation-induced pulmonary disorder.

### Leukocyte Infiltration

The difference in survival between the group that began receiving αCTGF mAb 2 days before or 2 days after irradiation was unexpected, since their treatment periods almost completely overlapped, with less than 1 week difference between them. The acute response to irradiation is characterized by edema and leukocyte infiltration, and CTGF has the potential to alter either or both of these events. CTGF has been reported to modulate the motility of immune cells such as macrophages, suggesting that αCTGF mAb could directly affect leukocyte infiltration. In addition, CTGF may alter secretion of chemokines and cytokines that recruit and maintain leukocytes. Therefore, CTGF may have direct and indirect effects on leukocyte infiltration that could be altered by the presence of αCTGF mAb before irradiation. To test this hypothesis, the number of leukocytes infiltrating the lungs at 2 days after irradiation (see Plathow et al. (2004) Invest. Radiol. 39:600-609) was compared for the irradiated and untreated group (IR) and the group that was pretreated with αCTGF mAb for 2 days prior to irradiation (d−2). A slightly smaller number of leukocytes per high power field were observed in lung slices from the αCTGF mAb-pretreated mice than from the irradiated, untreated mice (data not shown).

Leukocyte infiltration into the lungs of mice from all groups was also examined at 18 weeks after irradiation which corresponds to about the mid-point of the chronic response to irradiation. By 18 weeks, about 5 times the number of leukocytes was observed per field in the lungs of irradiated mice compared to unirradiated mice. Relative to irradiated and placebo-treated mice (IR+IgG), pretreatment of mice with αCTGF mAb (d−2) decreased leukocyte infiltration by a small but statistically significant amount, while treatment with αCTGF mAb beginning 2 days after irradiation (d+2) did not alter the number of leukocytes in the lungs at 18 weeks after administration. Administration of αCTGF mAb beginning 20 days or 16 weeks (112 days) after irradiation demonstrated a greater inhibition of leukocyte infiltration into the lungs at 18 weeks after irradiation, such that it was indistinguishable from that of unirradiated mice.

### Effect of αCTGF mAb on Irradiated Hearts

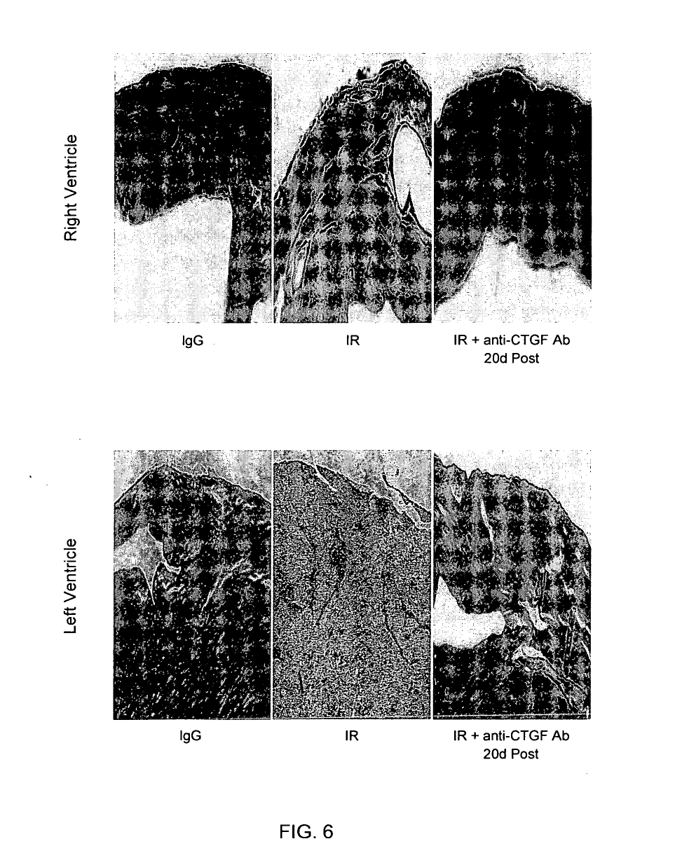

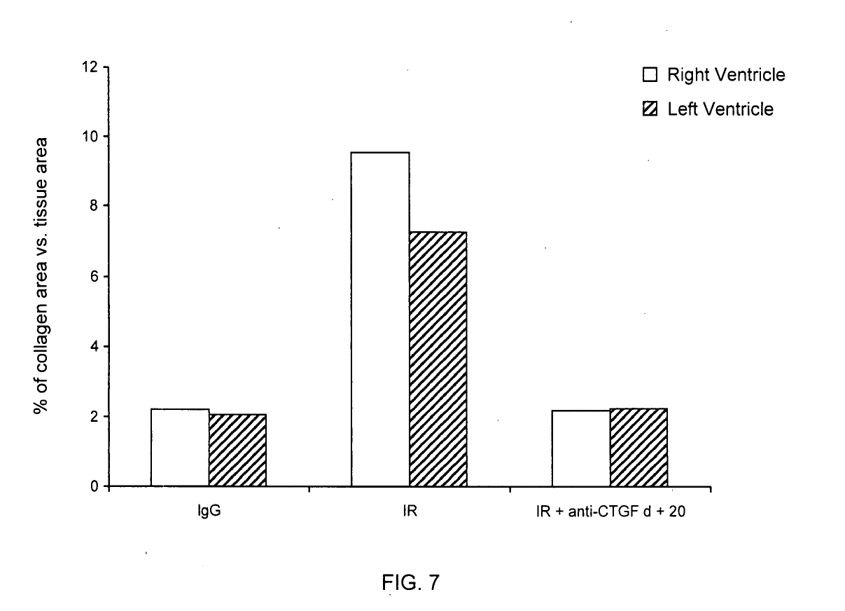

To examine the effect of irradiation on an organ other than the lung, the hearts of mice from some of the treatment groups were collected and fixed, and cross sections were stained with Sirius Red to visualize collagen (an indicator of fibrosis) (FIG. 6). To quantify collagen deposition, photographs of sections of the right and left ventricles were examined with Image-Pro Plus software (version 6.1; Media Cybernetics Inc., Bethesda, Md.) and the area represented by intense staining was expressed as a percent of total area (FIG. 7).

More collagen deposits could be observed in both the right ventricle (RV) and left ventricle (LV) of a mouse that was irradiated and untreated than in an unirradiated mouse exposed to the placebo antibody (IgG). Administration of αCTGF mAb for 8 weeks beginning 20 days after irradiation inhibited most of the radiation-induced deposition of collagen. Thus, treatment with the methods and agents of the present invention is effective in both lung and heart exposed to ionizing radiation.

Although the present examples were limited to 8 week treatment windows due to potential immune reaction in mice being treated with a human monoclonal antibody, the results clearly demonstrate that the methods provide significant improvement in all treatment periods. Thus, benefit can be seen in a subject whether the present methods are initiated prior to thoracic exposure to ionizing radiation or at times subsequent to exposure and even at times subsequent to the manifestation of acute or chronic effects of the radiation. The data clearly demonstrate that, to the extent possible, the methods should be initiated as early as possible and maintained throughout the period that the subject remains at risk for diminished organ function and compromised survivability. The data also clearly demonstrate that patients in the chronic progressive phase of the disorder can still benefit from the methods and medicaments of the present invention, improving lung function, reversing lung remodeling, reversing heart remodeling and improving survival.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed:

1. A method of treating a radiation-induced disorder in a subject having, or at risk of having, a radiation-induced disorder resulting from ionizing radiation exposure in the subject, the method comprising administering to the subject an anti-connective tissue growth factor (anti-CTGF) agent, thereby treating the disorder.

2. An anti-connective tissue growth factor (anti-CTGF) agent for use in treating a radiation-induced disorder in a subject having, or at risk of having, a radiation-induced disorder resulting from ionizing radiation exposure in the subject.

3. The method of claim 1, wherein the ionizing radiation exposure is a consequence of radiation therapy.

4. The method of claim 1, wherein the radiation-induced disorder is a disorder selected from the group consisting of a disorder of the lung, heart, bladder, large intestine, small intestine, gastrointestinal tract, esophagus, skin, kidney, head, neck, pancreas, liver, stomach, brain, spinal cord, urogenital system, prostate, testes, ovaries, vasculature, and muscle.

5. The method of claim 1, wherein the radiation-induced disorder is selected from the group consisting of radiation pneumonitis, radiation enteritis, radiation enteropathy, radiation enterocolitis, radiation dermatitis, radiation-induced erythema, radiation colitis, radiation proctitis, radiation cystitis, radiation nephritis, radiation esophagitis, radiation pericarditis, radiation-induced cardiac effusion, and radiation-induced cardiac fibrosis.

6. The method of claim 1, wherein the radiation-induced disorder results in impaired lung function in the subject and the method improves the impaired lung function.

7. The method of claim 6, wherein the impaired lung function is determined by an increase in lung density in the subject, and the improved lung function is determined by a decrease in lung density in the subject.

8. The method of claim 6, wherein the impaired lung function is determined by a decrease in fraction potential airspace in the subject, and the improved lung function is determined by an increase in fraction potential airspace in the subject.

9. The method of claim 6, wherein the impaired lung function is determined by a decrease in lung volume in the subject, and the improved lung function is determined by an increase in lung volume in the subject.

10. The method of claim 6, wherein the impaired lung function is determined by an increase in lung remodeling in the subject, and the improved lung function is determined by a decrease in lung remodeling in the subject

11. The method of claim 6, wherein the impaired lung function is determined by a decrease in PaO₂, in the subject, and the improved lung function is determined by an increase in PaO₂ in the subject.

12. The method of claim 1, wherein the subject has, or is at risk of having, a radiation-induced lung disorder resulting from thoracic exposure to ionizing radiation.

13. The method of claim 12, wherein the radiation-induced lung disorder is associated with pathological features selected from the group consisting of decreased lung volume, decreased fraction potential airspace, increased lung density, decreased PaO₂ and increased remodeled lung tissue.

14. A method of reducing, reversing, or stabilizing lung density in a subject having increased lung density associated with a radiation-induced lung disorder, the method comprising administering to the subject an anti-CTGF agent, thereby reducing or stabilizing lung density in the subject.

15. An anti-CTGF agent for use in reducing, reversing, or stabilizing lung density in a subject having increased lung density associated with a radiation-induced lung disorder.

16. A method of improving lung function in a subject having a radiation-induced lung disorder, the method comprising administering to the subject an anti-CTGF agent, thereby improving lung function in the subject.

17. An anti-CTGF agent for use in improving lung function in a subject having a radiation-induced lung disorder.

18. The method of claim 1, wherein the radiation-induced disorder is a radiation-induced heart disorder.

19. The method of claim 1, wherein the radiation-induced disorder is selected from a disorder from the group consisting of radiation pericarditis, radiation-induced cardiac effusion, and radiation-induced cardiac fibrosis.

20. An anti-CTGF agent for use in treating a radiation-induced heart disorder in a subject.

21. An anti-CTGF agent for use in treating a disorder in a subject selected from the group consisting of radiation pericarditis, radiation-induced cardiac effusion, and radiation-induced cardiac fibrosis.

22. The method or anti-CTGF agent of any one of the preceding claims, wherein the anti-CTGF agent is an anti-CTGF antibody.

23. The method or agent of claim 22, wherein the anti-CTGF antibody has the same amino acid sequence as the antibody produced by the cell line identified by ATCC Accession No. PTA-6006.

24. The method or agent of claim 22, wherein the anti-CTGF antibody binds to CTGF competitively with an antibody produced by the cell line identified by ATCC Accession No. PTA-6006.
